SEQUENCE LISTING (83 sequences)

SEQ ID NO: 1 - protein, 2 aa, Artificial sequence
  General cleavage motif recognized by Proprotein Convertases (PCs)
XX

SEQ ID NO: 2 - protein, 4 aa, Artificial sequence
  General cleavage motif recognized by PCs
XXXX

SEQ ID NO: 3 - protein, 6 aa, Artificial sequence
  General cleavage motif recognized by PCs
XXXXXX

SEQ ID NO: 4 - protein, 8 aa, Artificial sequence
  General cleavage motif recognized by PCs
XXXXXXXX

SEQ ID NO: 5 - protein, 7 aa, Artificial sequence
  Short CT sequence of ACE2
FTGIRDR

SEQ ID NO: 6 - protein, 4 aa, Artificial sequence
  Motif present in full-length CT of ACE2
YASI

SEQ ID NO: 7 - protein, 10 aa, Homo sapiens
CPSDSEEDEG

SEQ ID NO: 8 - protein, 4 aa, Homo sapiens
YKGL

SEQ ID NO: 9 - protein, 4 aa, Artificial sequence
  bait sequence for SKI-1
RXXX

SEQ ID NO: 10 - protein, 7 aa, Artificial sequence
  bait sequence for NARC-1/PCSK9
VFAQSIP

SEQ ID NO: 11 - protein, 34 aa, Homo sapiens
EKGTEAAGAMFLEAIPMSIPPEVKFNKPFVFLMI

SEQ ID NO: 12 - protein, 34 aa, Homo sapiens
EKGTEAAGAMFLERIPRSIPPEVKFNKPFVFLMI

SEQ ID NO: 13 - protein, 34 aa, Homo sapiens
EGTEAAAATGMAVRRKRAIMSPEEPIHFFADHPF

SEQ ID NO: 14 - protein, 12 aa, Homo sapiens
RKVFRSLKYAES

SEQ ID NO: 15 - protein, 12 aa, Homo sapiens
VTPQRKVFRSLK

SEQ ID NO: 16 - protein, 12 aa, Homo sapiens
RHSSRRLLRAIP

SEQ ID NO: 17 - protein, 12 aa, Homo sapiens
SGSGRSVLSFES

SEQ ID NO: 18 - protein, 12 aa, Homo sapiens
HSPGRNVLGTES

SEQ ID NO: 19 - protein, 12 aa, Homo sapiens
ANQRRHLLGFSA

SEQ ID NO: 20 - protein, 12 aa, Homo sapiens
GVLSRQLRALPS

SEQ ID NO: 21 - protein, 12 aa, Homo sapiens
VVRSRNLLIYEE

SEQ ID NO: 22 - protein, 12 aa, Mus musculus
QMPSRSLLFYDD

SEQ ID NO: 23 - protein, 12 aa, Homo sapiens
RVFSRTLHNDAA

SEQ ID NO: 24 - protein, 12 aa, Homo sapiens
KAGSRGLTSLAD

SEQ ID NO: 25 - protein, 12 aa, Rattus norvegicus
DPRLRQFLQKSL

SEQ ID NO: 26 - protein, 12 aa, Lassa virus
IYISRRLLGTFT

SEQ ID NO: 27 - protein, 12 aa, Crimean-Congo hemorrhagic fever virus
SSGSRRLLSEES

SEQ ID NO: 28 - protein, 12 aa, Lymphocytic choriomeningitis virus
KFLTRRLAGTFT

SEQ ID NO: 29 - protein, 10 aa, Artificial sequence
  bait sequence for Furin-like PCs
KRIRLRRSPD

SEQ ID NO: 30 - protein, 12 aa, Artificial sequence
  bait sequence for SKI-1/PCSK8
IYISRRLLGTFS

SEQ ID NO: 31 - protein, 11 aa, Artificial sequence
  bait sequence for NARC-1/PCSK9
SSVFAQSIPWN

SEQ ID NO: 32 - protein, 11 aa, Artificial sequence
  bait sequence for NARC-1/PCSK9
KHQKLLSIDLD

SEQ ID NO: 33 - protein, 10 aa, Artificial sequence
  bait sequence for BACE-APPsw
KISEVNLDAE

SEQ ID NO: 34 - protein, 10 aa, Artificial sequence
  bait sequence for BACE-APPsw (mut)
KISEVNFEVE

SEQ ID NO: 35 - protein, 4 aa, Artificial sequence
  convertase inhibitor
RVKR

SEQ ID NO: 36 - protein, 7 aa, Artificial sequence
  SKI-1 fluorogenic substrate
YISRRLL

SEQ ID NO: 37 - protein, 6 aa, Artificial sequence
  SKI-1 fluorogenic substrate
ISRRLL

SEQ ID NO: 38 - protein, 4 aa, Artificial sequence
  SKI-1 fluorogenic substrate
RRLL

SEQ ID NO: 39 - protein, 7 aa, Artificial sequence
  SKI-1 fluorogenic substrate
SGSRRLL

SEQ ID NO: 40 - protein, 7 aa, Artificial sequence
  SKI-1 fluorogenic substrate
FLTRRLS

SEQ ID NO: 41 - protein, 7 aa, Artificial sequence
  SKI-1 fluorogenic substrate
FFTRRLA

SEQ ID NO: 42 - protein, 7 aa, Artificial sequence
  convertase inhibitor
YISRRLL

SEQ ID NO: 43 - protein, 6 aa, Artificial sequence
  convertase inhibitor
ISRRLL

SEQ ID NO: 44 - protein, 4 aa, Artificial sequence
  convertase inhibitor
RRLL

SEQ ID NO: 45 - DNA, 906 bp, Artificial sequence
  encoding furin-like PCs short CT chimera
atggatcaattccgatggagaaggatgcctcgctggggactgctgctgctgctctggggctcctgtacctttggtctcccgacatacccctacgacgtgcccgactacgccgacaccaccacctttaaacggatcagactcagaagatcgccggatccggtcgagggtggaccatccgtcttcatcttccctccaaatatcaaggatgtactcatgatctccctgacacccaaggtcacgtgtgtggtggtggatgtgagcgaggatgacccagacgtccagatcagctggtttgtgaacaacgtggaagtacacacagctcagacacaaacccatagagaggattacaacagtactatccgggtggtcagcaccctccccatccagcaccaggactggatgagtggcaaggagttcaaatgcaaggtgaacaacaaagacctcccatcacccatcgagagaaccatctcaaaaattaaagggctagtcagagctccacaagtatacactttgccgccaccagcagagcagttgtccaggaaagatgtcagtctcacttgcctggtcgtgggcttcaaccctggagacatcagtgtggagtggaccagcaatgggcatacagaggagaactacaaggacaccgcaccagttcttgactctgacggttcttacctcatatatagcaagctcaatatgaaaacaagcaagtgggagaaaacagattccttctcatgcaacgtgagacacgagggtctgaaaaattactacctgaagaagaccatctcccggtctccgggtaaagatctgccccctgtttccatatggctgattgtttttggagttgtgatgggagtgatagtggttggcattgtcatcctgatcttcactgggatcagagatcggtag

SEQ ID NO: 46 - protein, 301 aa, Artificial sequence
  Furin-like PCs short CT chimera
MDQFRWRRMPRWGLLLLLWGSCTFGLPTYPYDVPDYADTTTFKRIRLRRSPDPVEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYTLPPPAEQLSRKDVSLTCLVVGFNPGDISVEWTSNGHTEENYKDTAPVLDSDGSYLIYSKLNMKTSKWEKTDSFSCNVRHEGLKNYYLKKTISRSPGKDLPPVSIWLIVFGVVMGVIVVGIVILIFTGIRDR

SEQ ID NO: 47 - DNA, 1017 bp, Artificial sequence
  encoding furin-like PCs full-length CT chimera
atggatcaattccgatggagaaggatgcctcgctggggactgctgctgctgctctggggctcctgtacctttggtctcccgacatacccctacgacgtgcccgactacgccgacaccaccacctttaaacggatcagactcagaagatcgccggatccggtcgagggtggaccatccgtcttcatcttccctccaaatatcaaggatgtactcatgatctccctgacacccaaggtcacgtgtgtggtggtggatgtgagcgaggatgacccagacgtccagatcagctggtttgtgaacaacgtggaagtacacacagctcagacacaaacccatagagaggattacaacagtactatccgggtggtcagcaccctccccatccagcaccaggactggatgagtggcaaggagttcaaatgcaaggtgaacaacaaagacctcccatcacccatcgagagaaccatctcaaaaattaaagggctagtcagagctccacaagtatacactttgccgccaccagcagagcagttgtccaggaaagatgtcagtctcacttgcctggtcgtgggcttcaaccctggagacatcagtgtggagtggaccagcaatgggcatacagaggagaactacaaggacaccgcaccagttcttgactctgacggttcttacctcatatatagcaagctcaatatgaaaacaagcaagtgggagaaaacagattccttctcatgcaacgtgagacacgagggtctgaaaaattactacctgaagaagaccatctcccggtctccgggtaaagatctgccccctgtttccatatggctgattgtttttggagttgtgatgggagtgatagtggttggcattgtcatcctgatcttcactgggatcagagatcggaagaagaaaaataaagcaagaagtggagaaaatccttatgcctccatcgatattagcaaaggagaaaataatccaggattccaaaacactgatgatgttcagacctccttttag

SEQ ID NO: 48 - protein, 338 aa, Artificial sequence
  Furin-like PCs full-length CT chimera
MDQFRWRRMPRWGLLLLLWGSCTFGLPTYPYDVPDYADTTTFKRIRLRRSPDPVEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYTLPPPAEQLSRKDVSLTCLVVGFNPGDISVEWTSNGHTEENYKDTAPVLDSDGSYLIYSKLNMKTSKWEKTDSFSCNVRHEGLKNYYLKKTISRSPGKDLPPVSIWLIVFGVVMGVIVVGIVILIFTGIRDRKKKNKARSGENPYASIDISKGENNPGFQNTDDVQTSF

SEQ ID NO: 49 - DNA, 918 bp, Artificial sequence
  encoding SKI-1 short CT chimera
atggatcaattccgatggagaaggatgcctcgctggggactgctgctgctgctctggggctcctgtacctttggtctcccgacatacccctacgacgtgcccgactacgccgacaccaccacctttatatacatctctagaaggctcctcggtacattcagtccggatccggtcgagggtggaccatccgtcttcatcttccctccaaatatcaaggatgtactcatgatctccctgacacccaaggtcacgtgtgtggtggtggatgtgagcgaggatgacccagacgtccagatcagctggtttgtgaacaacgtggaagtacacacagctcagacacaaacccatagagaggattacaacagtactatccgggtggtcagcaccctccccatccagcaccaggactggatgagtggcaaggagttcaaatgcaaggtgaacaacaaagacctcccatcacccatcgagagaaccatctcaaaaattaaagggctagtcagagctccacaagtatacactttgccgccaccagcagagcagttgtccaggaaagatgtcagtctcacttgcctggtcgtgggcttcaaccctggagacatcagtgtggagtggaccagcaatgggcatacagaggagaactacaaggacaccgcaccagttcttgactctgacggttcttacctcatatatagcaagctcaatatgaaaacaagcaagtgggagaaaacagattccttctcatgcaacgtgagacacgagggtctgaaaaattactacctgaagaagaccatctcccggtctccgggtaaagatctgccccctgtttccatatggctgattgtttttggagttgtgatgggagtgatagtggttggcattgtcatcctgatcttcactgggatcagagatcggtag

SEQ ID NO: 50 - protein, 305 aa, Artificial sequence
  SKI-1 short CT chimera
MDQFRWRRMPRWGLLLLLWGSCTFGLPTYPYDVPDYADTTTFIYISRRLLGTFSPDPVEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYTLPPPAEQLSRKDVSLTCLVVGFNPGDISVEWTSNGHTEENYKDTAPVLDSDGSYLIYSKLNMKTSKWEKTDSFSCNVRHEGLKNYYLKKTISRSPGKDLPPVSIWLIVFGVVMGVIVVGIVILIFTGIRDR

SEQ ID NO: 51 - DNA, 1029 bp, Artificial sequence
  encoding SKI-1 full-length CT chimera
atggatcaattccgatggagaaggatgcctcgctggggactgctgctgctgctctggggctcctgtacctttggtctcccgacatacccctacgacgtgcccgactacgccgacaccaccacctttatatacatctctagaaggctcctcggtacattcagtccggatccggtcgagggtggaccatccgtcttcatcttccctccaaatatcaaggatgtactcatgatctccctgacacccaaggtcacgtgtgtggtggtggatgtgagcgaggatgacccagacgtccagatcagctggtttgtgaacaacgtggaagtacacacagctcagacacaaacccatagagaggattacaacagtactatccgggtggtcagcaccctccccatccagcaccaggactggatgagtggcaaggagttcaaatgcaaggtgaacaacaaagacctcccatcacccatcgagagaaccatctcaaaaattaaagggctagtcagagctccacaagtatacactttgccgccaccagcagagcagttgtccaggaaagatgtcagtctcacttgcctggtcgtgggcttcaaccctggagacatcagtgtggagtggaccagcaatgggcatacagaggagaactacaaggacaccgcaccagttcttgactctgacggttcttacctcatatatagcaagctcaatatgaaaacaagcaagtgggagaaaacagattccttctcatgcaacgtgagacacgagggtctgaaaaattactacctgaagaagaccatctcccggtctccgggtaaagatctgccccctgtttccatatggctgattgtttttggagttgtgatgggagtgatagtggttggcattgtcatcctgatcttcactgggatcagagatcggaagaagaaaaataaagcaagaagtggagaaaatccttatgcctccatcgatattagcaaaggagaaaataatccaggattccaaaacactgatgatgttcagacctccttttag

SEQ ID NO: 52 - protein, 342 aa, Artificial sequence
  SKI-1 full-length CT chimera
MDQFRWRRMPRWGLLLLLWGSCTFGLPTYPYDVPDYADTTTFIYISRRLLGTFSPDPVEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYTLPPPAEQLSRKDVSLTCLVVGFNPGDISVEWTSNGHTEENYKDTAPVLDSDGSYLIYSKLNMKTSKWEKTDSFSCNVRHEGLKNYYLKKTISRSPGKDLPPVSIWLIVFGVVMGVIVVGIVILIFTGIRDRKKKNKARSGENPYASIDISKGENNPGFQNTDDVQTSF

SEQ ID NO: 53 - DNA, 921 bp, Artificial sequence
  encoding NARC short CT chimera
atggatcaattccgatggagaaggatgcctcgctggggactgctgctgctgctctggggctcctgtacctttggtctcccgacatacccctacgacgtgcccgactacgccgacaccaccacctttgaagaagactcctccgtgttcgctcagtccatcccgtggaacgatccggtcgagggtggaccatccgtcttcatcttccctccaaatatcaaggatgtactcatgatctccctgacacccaaggtcacgtgtgtggtggtggatgtgagcgaggatgacccagacgtccagatcagctggtttgtgaacaacgtggaagtacacacagctcagacacaaacccatagagaggattacaacagtactatccgggtggtcagcaccctccccatccagcaccaggactggatgagtggcaaggagttcaaatgcaaggtgaacaacaaagacctcccatcacccatcgagagaaccatctcaaaaattaaagggctagtcagagctccacaagtatacactttgccgccaccagcagagcagttgtccaggaaagatgtcagtctcacttgcctggtcgtgggcttcaaccctggagacatcagtgtggagtggaccagcaatgggcatacagaggagaactacaaggacaccgcaccagttcttgactctgacggttcttacctcatatatagcaagctcaatatgaaaacaagcaagtgggagaaaacagattccttctcatgcaacgtgagacacgagggtctgaaaaattactacctgaagaagaccatctcccggtctccgggtaaagatctgccccctgtttccatatggctgattgtttttggagttgtgatgggagtgatagtggttggcattgtcatcctgatcttcactgggatcagagatcggtag

SEQ ID NO: 54 - protein, 306 aa, Artificial sequence
  NARC short CT chimera
MDQFRWRRMPRWGLLLLLWGSCTFGLPTYPYDVPDYADTTTFEEDSSVFAQSIPWNDPVEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYTLPPPAEQLSRKDVSLTCLVVGFNPGDISVEWTSNGHTEENYKDTAPVLDSDGSYLIYSKLNMKTSKWEKTDSFSCNVRHEGLKNYYLKKTISRSPGKDLPPVSIWLIVFGVVMGVIVVGIVILIFTGIRDR

SEQ ID NO: 55 - protein, 13 aa, Artificial sequence
  bait sequence for NARC-1/PCSK9
EEDSSVAQSIPWN

SEQ ID NO: 56 - DNA, 1026 bp, Artificial sequence
  encoding NARC full-length CT chimera
atggatcaattccgatggagaaggatgcctcgctggggactgctgctgctgctctggggctcctgtacctttggtctcccgacatacccctacgacgtgcccgactacgccgacaccaccaccttttcctccgtgttcgctcagtccatcccgtggaacccggatccggtcgagggtggaccatccgtcttcatcttccctccaaatatcaaggatgtactcatgatctccctgacacccaaggtcacgtgtgtggtggtggatgtgagcgaggatgacccagacgtccagatcagctggtttgtgaacaacgtggaagtacacacagctcagacacaaacccatagagaggattacaacagtactatccgggtggtcagcaccctccccatccagcaccaggactggatgagtggcaaggagttcaaatgcaaggtgaacaacaaagacctcccatcacccatcgagagaaccatctcaaaaattaaagggctagtcagagctccacaagtatacactttgccgccaccagcagagcagttgtccaggaaagatgtcagtctcacttgcctggtcgtgggcttcaaccctggagacatcagtgtggagtggaccagcaatgggcatacagaggagaactacaaggacaccgcaccagttcttgactctgacggttcttacctcatatatagcaagctcaatatgaaaacaagcaagtgggagaaaacagattccttctcatgcaacgtgagacacgagggtctgaaaaattactacctgaagaagaccatctcccggtctccgggtaaagatctgccccctgtttccatatggctgattgtttttggagttgtgatgggagtgatagtggttggcattgtcatcctgatcttcactgggatcagagatcggaagaagaaaaataaagcaagaagtggagaaaatccttatgcctccatcgatattagcaaaggagaaaataatccaggattccaaaacactgatgatgttcagacctccttttag

SEQ ID NO: 57 - protein, 341 aa, Artificial sequence
  NARC full-length CT chimera
MDQFRWRRMPRWGLLLLLWGSCTFGLPTYPYDVPDYADTTTFSSVFAQSIPWNPDPVEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYTLPPPAEQLSRKDVSLTCLVVGFNPGDISVEWTSNGHTEENYKDTAPVLDSDGSYLIYSKLNMKTSKWEKTDSFSCNVRHEGLKNYYLKKTISRSPGKDLPPVSIWLIVFGVVMGVIVVGIVILIFTGIRDRKKKNKARSGENPYASIDISKGENNPGFQNTDDVQTSF

SEQ ID NO: 58 - DNA, 1026 bp, Artificial sequence
  encoding NARC full-length CT chimera
atggatcaattccgatggagaaggatgcctcgctggggactgctgctgctgctctggggctcctgtacctttggtctcccgacatacccctacgacgtgcccgactacgccgacaccaccacctttaaacatcagaagctactctccattgacctggacccggatccggtcgagggtggaccatccgtcttcatcttccctccaaatatcaaggatgtactcatgatctccctgacacccaaggtcacgtgtgtggtggtggatgtgagcgaggatgacccagacgtccagatcagctggtttgtgaacaacgtggaagtacacacagctcagacacaaacccatagagaggattacaacagtactatccgggtggtcagcaccctccccatccagcaccaggactggatgagtggcaaggagttcaaatgcaaggtgaacaacaaagacctcccatcacccatcgagagaaccatctcaaaaattaaagggctagtcagagctccacaagtatacactttgccgccaccagcagagcagttgtccaggaaagatgtcagtctcacttgcctggtcgtgggcttcaaccctggagacatcagtgtggagtggaccagcaatgggcatacagaggagaactacaaggacaccgcaccagttcttgactctgacggttcttacctcatatatagcaagctcaatatgaaaacaagcaagtgggagaaaacagattccttctcatgcaacgtgagacacgagggtctgaaaaattactacctgaagaagaccatctcccggtctccgggtaaagatctgccccctgtttccatatggctgattgtttttggagttgtgatgggagtgatagtggttggcattgtcatcctgatcttcactgggatcagagatcggaagaagaaaaataaagcaagaagtggagaaaatccttatgcctccatcgatattagcaaaggagaaaataatccaggattccaaaacactgatgatgttcagacctccttttag

SEQ ID NO: 59 - protein, 341 aa, Artificial sequence
  NARC full-length CT chimera
MDQFRWRRMPRWGLLLLLWGSCTFGLPTYPYDVPDYADTTTFKHQKLLSIDLDPDPVEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYTLPPPAEQLSRKDVSLTCLVVGFNPGDISVEWTSNGHTEENYKDTAPVLDSDGSYLIYSKLNMKTSKWEKTDSFSCNVRHEGLKNYYLKKTISRSPGKDLPPVSIWLIVFGVVMGVIVVGIVILIFTGIRDRKKKNKARSGENPYASIDISKGENNPGFQNTDDVQTSF

SEQ ID NO: 60 - DNA, 912 bp, Artificial sequence
  encoding BACE-APP sw short CT chimera
atggatcaattccgatggagaaggatgcctcgctggggactgctgctgctgctctggggctcctgtacctttggtctcccgacatacccctacgacgtgcccgactacgccgacaccaccacctttaaaatctctgaagtgaacctcgatgcagaaccggatccggtcgagggtggaccatccgtcttcatcttccctccaaatatcaaggatgtactcatgatctccctgacacccaaggtcacgtgtgtggtggtggatgtgagcgaggatgacccagacgtccagatcagctggtttgtgaacaacgtggaagtacacacagctcagacacaaacccatagagaggattacaacagtactatccgggtggtcagcaccctccccatccagcaccaggactggatgagtggcaaggagttcaaatgcaaggtgaacaacaaagacctcccatcacccatcgagagaaccatctcaaaaattaaagggctagtcagagctccacaagtatacactttgccgccaccagcagagcagttgtccaggaaagatgtcagtctcacttgcctggtcgtgggcttcaaccctggagacatcagtgtggagtggaccagcaatgggcatacagaggagaactacaaggacaccgcaccagttcttgactctgacggttcttacctcatatatagcaagctcaatatgaaaacaagcaagtgggagaaaacagattccttctcatgcaacgtgagacacgagggtctgaaaaattactacctgaagaagaccatctcccggtctccgggtaaagatctgccccctgtttccatatggctgattgtttttggagttgtgatgggagtgatagtggttggcattgtcatcctgatcttcactgggatcagagatcggtag

SEQ ID NO: 61 - protein, 303 aa, Artificial sequence
  BACE-APP sw short CT chimera
MDQFRWRRMPRWGLLLLLWGSCTFGLPTYPYDVPDYADTTTFKISEVNLDAEPDPVEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYTLPPPAEQLSRKDVSLTCLVVGFNPGDISVEWTSNGHTEENYKDTAPVLDSDGSYLIYSKLNMKTSKWEKTDSFSCNVRHEGLKNYYLKKTISRSPGKDLPPVSIWLIVFGVVMGVIVVGIVILIFTGIRDR

SEQ ID NO: 62 - DNA, 1023 bp, Artificial sequence
  encoding BACE-APP sw full-length CT chimera
atggatcaattccgatggagaaggatgcctcgctggggactgctgctgctgctctggggctcctgtacctttggtctcccgacatacccctacgacgtgcccgactacgccgacaccaccacctttaaaatctctgaagtgaacctcgatgcagaaccggatccggtcgagggtggaccatccgtcttcatcttccctccaaatatcaaggatgtactcatgatctccctgacacccaaggtcacgtgtgtggtggtggatgtgagcgaggatgacccagacgtccagatcagctggtttgtgaacaacgtggaagtacacacagctcagacacaaacccatagagaggattacaacagtactatccgggtggtcagcaccctccccatccagcaccaggactggatgagtggcaaggagttcaaatgcaaggtgaacaacaaagacctcccatcacccatcgagagaaccatctcaaaaattaaagggctagtcagagctccacaagtatacactttgccgccaccagcagagcagttgtccaggaaagatgtcagtctcacttgcctggtcgtgggcttcaaccctggagacatcagtgtggagtggaccagcaatgggcatacagaggagaactacaaggacaccgcaccagttcttgactctgacggttcttacctcatatatagcaagctcaatatgaaaacaagcaagtgggagaaaacagattccttctcatgcaacgtgagacacgagggtctgaaaaattactacctgaagaagaccatctcccggtctccgggtaaagatctgccccctgtttccatatggctgattgtttttggagttgtgatgggagtgatagtggttggcattgtcatcctgatcttcactgggatcagagatcggaagaagaaaaataaagcaagaagtggagaaaatccttatgcctccatcgatattagcaaaggagaaaataatccaggattccaaaacactgatgatgttcagacctccttttag

SEQ ID NO: 63 - protein, 340 aa, Artificial sequence
  BACE-APP sw full-length CT chimera
MDQFRWRRMPRWGLLLLLWGSCTFGLPTYPYDVPDYADTTTFKISEVNLDAEPDPVEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYTLPPPAEQLSRKDVSLTCLVVGFNPGDISVEWTSNGHTEENYKDTAPVLDSDGSYLIYSKLNMKTSKWEKTDSFSCNVRHEGLKNYYLKKTISRSPGKDLPPVSIWLIVFGVVMGVIVVGIVILIFTGIRDRKKKNKARSGENPYASIDISKGENNPGFQNTDDVQTSF

SEQ ID NO: 64 - DNA, 912 bp, Artificial sequence
  encoding BACE-APP sw (mut) short CT chimera
atggatcaattccgatggagaaggatgcctcgctggggactgctgctgctgctctggggctcctgtacctttggtctcccgacatacccctacgacgtgcccgactacgccgacaccaccacctttaaaatctctgaagtgaacttcgaagtagaaccggatccggtcgagggtggaccatccgtcttcatcttccctccaaatatcaaggatgtactcatgatctccctgacacccaaggtcacgtgtgtggtggtggatgtgagcgaggatgacccagacgtccagatcagctggtttgtgaacaacgtggaagtacacacagctcagacacaaacccatagagaggattacaacagtactatccgggtggtcagcaccctccccatccagcaccaggactggatgagtggcaaggagttcaaatgcaaggtgaacaacaaagacctcccatcacccatcgagagaaccatctcaaaaattaaagggctagtcagagctccacaagtatacactttgccgccaccagcagagcagttgtccaggaaagatgtcagtctcacttgcctggtcgtgggcttcaaccctggagacatcagtgtggagtggaccagcaatgggcatacagaggagaactacaaggacaccgcaccagttcttgactctgacggttcttacctcatatatagcaagctcaatatgaaaacaagcaagtgggagaaaacagattccttctcatgcaacgtgagacacgagggtctgaaaaattactacctgaagaagaccatctcccggtctccgggtaaagatctgccccctgtttccatatggctgattgtttttggagttgtgatgggagtgatagtggttggcattgtcatcctgatcttcactgggatcagagatcggtag

SEQ ID NO: 65 - protein, 303 aa, Artificial sequence
  BACE-APP sw (mut) short CT chimera
MDQFRWRRMPRWGLLLLLWGSCTFGLPTYPYDVPDYADTTTFKISEVNFEVEPDPVEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYTLPPPAEQLSRKDVSLTCLVVGFNPGDISVEWTSNGHTEENYKDTAPVLDSDGSYLIYSKLNMKTSKWEKTDSFSCNVRHEGLKNYYLKKTISRSPGKDLPPVSIWLIVFGVVMGVIVVGIVILIFTGIRDR

SEQ ID NO: 66 - DNA, 1023 bp, Artificial sequence
  encoding BACE-APP sw (mut) full-length CT chimera
atggatcaattccgatggagaaggatgcctcgctggggactgctgctgctgctctggggctcctgtacctttggtctcccgacatacccctacgacgtgcccgactacgccgacaccaccacctttaaaatctctgaagtgaacttcgaagtagaaccggatccggtcgagggtggaccatccgtcttcatcttccctccaaatatcaaggatgtactcatgatctccctgacacccaaggtcacgtgtgtggtggtggatgtgagcgaggatgacccagacgtccagatcagctggtttgtgaacaacgtggaagtacacacagctcagacacaaacccatagagaggattacaacagtactatccgggtggtcagcaccctccccatccagcaccaggactggatgagtggcaaggagttcaaatgcaaggtgaacaacaaagacctcccatcacccatcgagagaaccatctcaaaaattaaagggctagtcagagctccacaagtatacactttgccgccaccagcagagcagttgtccaggaaagatgtcagtctcacttgcctggtcgtgggcttcaaccctggagacatcagtgtggagtggaccagcaatgggcatacagaggagaactacaaggacaccgcaccagttcttgactctgacggttcttacctcatatatagcaagctcaatatgaaaacaagcaagtgggagaaaacagattccttctcatgcaacgtgagacacgagggtctgaaaaattactacctgaagaagaccatctcccggtctccgggtaaagatctgccccctgtttccatatggctgattgtttttggagttgtgatgggagtgatagtggttggcattgtcatcctgatcttcactgggatcagagatcggaagaagaaaaataaagcaagaagtggagaaaatccttatgcctccatcgatattagcaaaggagaaaataatccaggattccaaaacactgatgatgttcagacctccttttag

SEQ ID NO: 67 - protein, 340 aa, Artificial sequence
  BACE-APP sw (mut) full-length CT chimera
MDQFRWRRMPRWGLLLLLWGSCTFGLPTYPYDVPDYADTTTFKISEVNFEVEPDPVEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYTLPPPAEQLSRKDVSLTCLVVGFNPGDISVEWTSNGHTEENYKDTAPVLDSDGSYLIYSKLNMKTSKWEKTDSFSCNVRHEGLKNYYLKKTISRSPGKDLPPVSIWLIVFGVVMGVIVVGIVILIFTGIRDRKKKNKARSGENPYASIDISKGENNPGFQNTDDVQTSF

SEQ ID NO: 68 - protein, 28 aa, Artificial sequence
  Renin signal peptide
MDQFRWRRMPRWGLLLLLWGSCTFGLPT

SEQ ID NO: 69 - protein, 9 aa, Artificial sequence
  Hemaglutinin A (HA) Tag
YPYDVPDYA

SEQ ID NO: 70 - protein, 214 aa, Artificial sequence
  Fc fragment of immunoglobulin
EGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYTLPPPAEQLSRKDVSLTCLVVGFNPGDISVEWTSNGHTEENYKDTAPVLDSDGSYLIYSKLNMKTSKWEKTDSFSCNVRHEGLKNYYLKKTISRSPGKD

SEQ ID NO: 71 - protein, 25 aa, Artificial sequence
  ACE-2 transmembrane domain
PPVSIWLIVFGVVMGVIVVGIVILI

SEQ ID NO: 72 - protein, 44 aa, Artificial sequence
  Full-length CT of ACE2
FTGIRDRKKKNKARSGENPYASIDISKGENNPGFQNTDDVQTSF

SEQ ID NO: 73 - protein, 14 aa, Artificial sequence
  HA Tag with linker sequence
YPYDVPDYADTTTF

SEQ ID NO: 74 - protein, 5 aa, Artificial sequence
  Linker sequence
DTTTF

SEQ ID NO: 75 - protein, 10 aa, Artificial sequence
  Bait sequence for PC5
KRIRLRRLPD

SEQ ID NO: 76 - protein, 4 aa, Artificial sequence
  Fluorogenic substrate for Furin-like PCs
RVKR

SEQ ID NO: 77 - protein, 4 aa, Artificial sequence
  NARC-1 substrate motif
VFAQ

SEQ ID NO: 78 - protein, 4 aa, Artificial sequence
  furin substrate motif
RERR

SEQ ID NO: 79 - protein, 4 aa, Artificial sequence
  furin and PC5 substrate motif
RKKR

SEQ ID NO: 80 - protein, 4 aa, Artificial sequence
  SKI-1 substrate motif
RRLX

SEQ ID NO: 81 - protein, 6 aa, Artificial sequence
  alpha-secretase substrate motif
HHQKLV

SEQ ID NO: 82 - protein, 6 aa, Artificial sequence
  beta-secretase substrate motif
EVKMDA

SEQ ID NO: 83 - protein, 4 aa, Artificial sequence
  endoplasmic reticulum retention signal
KDEL

(19) United States
(12) Patent Application Publication
(10) Pub. No.: US 2009/0130691 A1
(43) Pub. Date: May 21, 2009

(54) SCREENING PROTEINASE MODULATORS USING A CHIMERIC PROTEIN AND SKI-I PROPROTEIN CONVERTASE SUBSTRATES AND INHIBITORS

(75) Inventors: Nabil Seidah, Ile Des Soeurs (CA); Timothy L. Reudelhuber, Baie d' Urfe (CA)

Correspondence Address: GOUDREAU GAGE DUBUC, 2000 MCGILL COLLEGE, SUITE 2200, MONTREAL, QC, H3A 3H3, CA

(73) Assignee: Institut de Recherches Cliniques de Montreal, Montreal, QC (CA)

(21) Appl. No.: 12/066,788

(22) PCT Filed: Sep. 14, 2006
(86) PCT No.: PCT/CA2006/001514

§ 371 Date: Mar. 13, 2008

Related U.S. Application Data
(60) Provisional application No. 60/717,254, filed on Sep. 16, 2005.

(51) Int. Cl. G01N 33/53; C07K 16/18; C12N 5/06; C07K 7/00; C12Q 1/37; C12Q 1/42

(52) U.S. Cl. 435/7.2; 530/387.3; 435/328; 435/23; 435/21; 530/329

(57) ABSTRACT

A chimeric protein comprising in sequence a signal peptide, a first amino acid tag, a proteinase bait, a second amino acid tag, a transmembrane domain and a cytosolic domain, wherein the cytosolic (CT) domain comprises a sequence able to recycle the protein from the cellular membrane to endosomes. A cell line expressing the chimeric protein and an assay using the cell line.

### CROSS REFERENCE TO RELATED APPLICATIONS

This application claims priority on U.S. provisional application No. 60/717,254, filed on Sep. 16, 2005. All documents above are herein incorporated by reference.

### FIELD OF THE INVENTION

The present invention relates to chimeric proteins, cell lines comprising same, and assays for screening proteinase modulators using same. More specifically, the present invention is concerned with cell-based assays for identifying modulators of constitutively secreted proteinases, chimeric proteins for use therein and cells expressing the chimeric proteins.

### BACKGROUND OF THE INVENTION

### Processing and Activation of PCs Secretory Precursors

The Proprotein Convertases (PCs) are responsible for the tissue-specific limited proteolysis of multiple polypeptide precursors, generating a large diversity of bioactive molecules (Seidah and Chretien, 1999; Seidah and Prat, 2002). Secretory precursors are usually cleaved at the general motif (K/R)-(X)n-(K/R)←, where n=0 (SEQ ID NO: 1), 2 (SEQ ID NO: 2), 4 (SEQ ID NO: 3) or 6 (SEQ ID NO: 4) and X is usually not a Cys. Seven dibasic-specific PCs, serine proteinases belonging to the kexin subfamily of subtilases, were identified: Furin, PC1 (also called PC3), PC2, PC4, PACE4, PC5 (also called PC6), and PC7 (also called PC8, LPC or SPC7) (Seidah and Chretien, 1999; Seidah and Prat, 2002). These enzymes are inhibited by their prosegments (Zhong et al., 1999; Nour et al., 2003b) and by the serpin variant α1-PDX (Benjannet et al., 1997; Anderson et al., 1993). The Applicant's group also identified two non basic-aa-specific PCs, subtilisin-kexin isoenzyme (SKI-1/PCSK8/S1P) and NARC-1/PCSK9. They belong to the Pyrolysin and Proteinase K subfamily of subtilases, respectively. While SKI-1 exhibits a cleavage specificity for a (R/K)-X-(hydrophobic)-(L,T)← (Brown and Goldstein, 1999; Seidah et al., 1999; Toure et al., 2000) so far NARC-1/PCSK9 seems to prefer the VFAQ← motif (Seidah et al., 2003; Benjannet et al., 2004b).

The Regulation of Processing within the Constitutive Secretory Pathway

Many cellular processing events involve an ordered cascade of cleavage events accomplished by one or more convertase(s) belonging to the PCs/SKI-1/NARC-1 mammalian subtilase family (Seidah, 2002; Seidah and Prat, 2002; Seidah et al., 2003). A number of factors regulate this ordered process. First, PCs require removal of their inhibitory prosegment for activation. Analysis of the biosynthesis of Furin, PACE4; PC5; PC7, SKI-1, and NARC-1 revealed that they are synthesized as zymogens that undergo autocatalytic cleavage of their N-terminal inhibitory pro-segment, which seems to act both as a chaperone and an intramolecular inhibitor. Overexpression of Furin, PC5, and PC7 prosegments as independent domains confirmed their inhibitory potency and the presence of critical elements at their C-terminus. The second control element is the trafficking of these enzymes to different intracellular organelles. Cellular localization experiments revealed that SKI-1 is sorted to the cis/medial Golgi (“Golgi”), suggesting that it is poised to process its cognate precursors, SREBPs, ATF6, proBDNF, Luman, and somatostatin before the dibasic-specific PCs. Dependant on the cognate substrate, constitutively secreted PCs cleave in the Golgi, the TGN, the endosomes or the cell surface. The modified serpin α1-PDX (Benjannet et al., 1997; Anderson et al., 1993) and the PC-prosegments (Zhong et al., 1999) inhibit the PCs within the constitutive secretory pathway. The design of two potent and specific-inhibitors of SKI-1 based on variants of either its prosegment or α1-PDX were reported (Pullikotil et al., 2004). Although no NARC-1 inhibitor is yet known, potent NARC-1 siRNAs were identified that upregulate the LDLR (Benjannet et al., 2004b).

### Implication of PCs in Activation of Viral Glycoproteins

Data on various infectious viruses and bacterial toxins showed that cleavage of surface precursors by one or more PC is a required step for the acquisition of fusiogenic potential and thus for infectious capacity of viral particles and bacterial toxins. Studies demonstrated that Furin and PC7 cleave HIV-1 gp160 and other viral surface glycoproteins. For most retroviral glycoproteins cleavage occurs C-terminal to single or pairs of basic residues within the consensus motif (R/K)-(X)n-(K/R)←, [where n=0, 2, 4, or 6] (Seidah and Chretien, 1999; Seidah et al., 1998b; Seidah and Chretien, 1997). In the Hong Kong influenza virus, an RERR insertion N-terminal to RKKR1 sequence results in a ˜5-fold increase in cleavage efficacy by Furin or PC5 and contributes to the high viral infectivity. The surface glycoproteins of several hemorrhagic fever viruses such as Lassa, Crimean Congo hemorrhagic fever and Lymphocytic Choriomeningitis were shown to be cleaved by SKI-1 at RRLX← motifs. The implication of the PCs in the viral infection and spread of SARS-CoV that severely affected Canada last year, resulting in 44 death was also recently reported (Bergeron et al., 2005).

### Implication of PCs in Cancer/Metastasis

Some of the protein precursors cleaved by the PCs, such as matrix metalloproteases, adhesion molecules, growth factors, and growth factor receptors are directly or indirectly involved in tumorigenesis and metastasis by regulating either degradation of extra-cellular matrix and/or modulation of cell growth and survival (Khatib et al., 2001; Khatib et al., 2002). It was shown that expression of the PC-inhibitor α1-PDX in metastatic colon cancer cells, when inoculated in nude mice, resulted in decreased invasion, lower incidence of tumor development, with a significantly reduced tumor size and vascularization. Using various PC inhibitors and site-directed mutagenesis, PDGF-A, PDGF-B and VEGF-C were identified as new substrates for the PCs. This highlighted the importance of PCs in the activation of these growth factors during tumor progression and angiogenesis (Siegfried et al., 2003a; Siegfried et al., 2003b; Siegfried et al., 2005).

### Implication of Proteinase in Neurodegenerative Pathologies, e.g., Alzheimer's Disease

The proteins βAPP, presenilin 1 (PS1) and PS2 have been implicated in the early-onset of autosomal dominant Alzheimer's disease (AD). Intense efforts have been directed toward the identification of the secretases involved in the processing of βAPP, termed α-, β- and γ-secretases. The PCs process the zymogens of both α- and β-secretases.

α-secretase cleaves βAPP at the HHQK₆₆₈←LV sequence resulting in non-amyloidogenic products (sAPα). ADAM10 and ADAM17 were reported to be involved in the α-cleavage of βAPP. It was demonstrated that inhibition of PCs by α1-PDX blocks the α-secretase cleavage of βAPP, while overexpression of PC7 enhances it (Lopez-Perez et al., 1999), PC7 and ADAM10, but not ADAM17, likely contribute to the constitutive secretion of soluble sAPPα by human LoVo cells (Lopez-Perez et al., 2001).

β-secretase (BACE): The amyloidogenic pathway generating Aβ starts by the β-secretase cleavage of βAPP at the EVKM₆₅₂←DA sequence. The major β-secretase candidate is BACE, a type-I membrane-bound aspartyl proteinase that is constitutively secreted. It was reported that Furin and PC5A are the major PCs responsible for the conversion of proBACE into BACE within the TGN (Benjannet et al., 2001). Some of BACE singularities are its palmitoylation at the three Cys residues and the Ser-phosphorylation within its cytosolic tail and its sulfation at one or more of its carbohydrate moieties. It was shown that BACE undergoes metabolic processing at specific Asp residues resulting in multiple forms (Benjannet et al., 2004a). Using a yeast two hybrid system, 7 brain-specific interactors with the cytosolic tail of BACE were identified, including BRI-3, and it was shown that both BACE and BRI-3 are processed by Furin (Wickham et al., 2005).

### Consequences of PC Knockout

Knockout (KO) of PC1, PC2, PC4, PACE4, or PC7 genes resulted in viable animals with relatively mild phenotypes. In contrast, Furin KO mice are embryonic lethal, whereas conditional KO of Furin in liver results in viable mice. Contrary to two reported cases of human PC1 KO patients who exhibit either obesity or severe digestion problems, PC1 KO mice have a retarded growth phenotype, likely due to the absence of the PC1 cleavage of proGHRH. PC2-null mice are mildly diabetic and runted and the processing of some hormonal peptide precursor is affected. Interestingly, 7B2 KO mice, which lack PC2 activity, die within three weeks after birth from severe Cushing's disease due to excessive secretion of ACTH by the intermediate lobe of the pituitary. It is important to note that although both PC2 and 7B2 KO mice lack PC2 activity, their phenotypes are widely different, in part possibly related to the use of different mouse genetic backgrounds. It was also found that PC5 KO homozygote are embryonic lethal, where death occurs before e7.5. SKI-1 nulls were also found to be embryonic lethal and a conditional SKI-1 KO in liver clearly emphasized the role of this convertase in cholesterol and lipid metabolism. Thus, Furin, PC5, SKI-1 have been shown to be embryonic lethal in mice. Very recently, three human families exhibiting dominant hypercholesterolemia (FH3) were found to be heterozygote for two different NARC-1 mutations in the coding region (Abifadel et al., 2003). These and other new natural mutations were biochemically analyzed and it was shown that they result in a gain of function (Benjannet et al., 2004b). This is the first dominant human pathology directly associated to mutations in a PC. Interestingly, two heterozygote loss of function stop mutations found in African Americans result in an opposite phenotype, namely hypocholesterolemia (Cohen et al., 2005).

### Known Assays to Identify PCs Inhibitors

Most of the in vitro assays designed for identifying proteinase inhibitory molecules consist in the addition of a compound to a reaction mixture containing a purified enzyme (e.g. SKI-1) and a substrate (e.g. CREB-H protein), and measuring the absence or reduction of the cleavage products observed when the mixture is incubated under similar conditions but without the inhibitory compound. Such in vitro assays do not select for compounds able to penetrate the cell nor for compounds able to reach and be effective in the cellular compartments supporting the constitutive secretory pathway.

There is thus a need for modulators of proteinase activities. However, some inhibitors active in vitro may not find utility in vivo because they do not reach the cellular proteinase.

Cell-based assays were thus devised. However prior art cell-based assays for identifying convertase-inhibitory compounds produced false positives. Oh et al. 2004 described a cell-based assay for β-secretase (BACE) activity using a target chimeric protein substrate containing three domains: an amino-terminal TM domain, a beta-site and an alkaline phosphatase (ALP). In this assay, the activity of BACE on the chimera results in the release of ALP in the culture medium. An inhibition of the BACE activity results in the absence of ALP release in the culture medium. However, an absence of ALP in the culture medium could result not only from the inhibition of the target substrate cleavage itself, but also from a variety of irrelevant cellular mechanisms including amongst others, the absence of target chimeric protein substrate expression itself, modification of chaperones, cellular trafficking, protein folding or even a pH change within the cells, etc. It was thus difficult to determine through their use whether the absence of detection of a specific signal resulted from the convertase inactivation or from another irrelevant reason.

A positive cell-based assays targeting a subcellular compartment and used for the identification of protease inhibitors was also described (Belkhirin et al. 2002). This assay, which targets cathepsin L in the lysosome, is however not appropriate for the identification of inhibitors of PCs in the TGN, endosomes or cell surface.

There is thus a need for new cell-based assays that would provide fewer false positives. There is also a need for cell-based assays useful for PCs and other proteinases.

There is thus a need for improved cell-based assays for identifying proteinase inhibitors.

The present invention seeks to meet these needs and other needs.

The present description refers to a number of documents, the content of which is herein incorporated by reference in their entirety.

### SUMMARY OF THE INVENTION

Five classes of proteinases are known, including the Serine (Ser), Aspartic (Asp), Metallo, Cysteine (Cys) and Threonine (Thr) proteinases, and are estimated to contain a total of 500-600 members in the human and mouse genomes. Different proteinases digest their substrates within different specific cell compartments or extracellularly. For instance, the proteinases present in the proteasome (Certain Ser, Asp, and Thr proteinases) are active throughout the cytoplasm and the nucleus, caspases (Cys proteinases) are active in the cytoplasm, and still other proteinases are active in the secretory and endocytic pathways.

The secretory and endocytic pathways of eukaryotic organelles consist of multiple compartments. Specific transport mechanisms are required to direct molecules to defined locations. The localization of proteins to specific membranes is complex and involves multiple interactions. All of the proteins that pass through the Golgi apparatus, except those that are retained there as permanent residents, are sorted in the trans Golgi network (TGN) according to their intended final destination. The terminology “secretory and endocytic pathways” is a generic term covering various pathways including the pathway of proteins sorted to lysosomes (e.g. cathepsin B), the pathway of proteins recycled into earlier secretory compartments by recognition of a retention signal (e.g. KDEL for the endoplasmic reticulum), the regulatory pathway and the constitutive secretory pathway.

The regulatory pathway is one by which a specific subset of proteins of certain endocrine and neuroendocrine cells are sorted from the TGN to electron-dense cytoplasmic granules (dense core secretory granules) where they are stored until the cell receives a signal for their release. This pathway depends on a highly selective triage of proteins within or just after the TGN within immature secretory granules. Some PCs, namely PC1, PC2 and sometimes PC5A are sorted to the regulatory pathway.

The constitutive secretory pathway, is one by which proteins are secreted from the cells at a rate that is only limited by their rate of synthesis. These proteins follow a pathway that goes through the endoplasmic reticulum (ER), the Golgi, the TGN and finally through the cell surface. Some of the constitutively secreted proteins however could once at the cell surface be re-internalized via early endosomes and then directed towards either 1) the TGN once again, 2) lysosomes; or even 3) be recycled to the cell surface for another round of sorting. This trafficking is intimately associated with sorting motifs found within the cytoplasmic tail of these usually membrane-bound proteins. PCs including Furin, PC4, PC5B, PC7, PACE4, SKI-1 and NARC-1/PCSK9, along with aspartyl proteinase such as BACE and MMPs are mostly sorted through the constitutive secretory pathway.

Depending on the cognate substrate, constitutively secreted PCs and other constitutively secreted proteinases may cleave in the Golgi, the TGN, the endosomes, the cell surface or a combination of these locations. Specifically, Furin, PC7, PACE4 and PC5B cleave precursors within the TGN, endosomes and cell surface. SKI-1 cleaves the transcription factors (SREBPs and ATF6) in the medial Golgi or cell surface. However, it cleaves itself into a soluble form within endosomes. BACE cleaves mostly in the TGN and endosomes. NARC-1/PCSK9 seems to enhance the degradation of the LDLR within acidic compartments, likely to be clathrin coated endosomes.

MMPs can either be membrane bound (MT-MMPs) or soluble. The membrane bound forms are recycled. However, the optimal pH for MT-MMPs is usually neutral and MT-MMPs are thus thought to act at or near the cell surface.

The present invention provides cell-based assays for monitoring a cellular proteinase activity and modulators thereof that are effective in the constitutive secretory pathway. The assay can be adapted to isolate inhibitors or activators of a selected proteinase. If the cell line used has all its bait sequence initially cleaved off, it may be used to screen proteinase inhibitors: the presence of inhibitors would be detected by a reappearance of cell-surface expressed baits. Conversely, if the cell line used still has detectable levels of the bait left uncleaved, it can be used to screen proteinase activators: the presence of activators would be detected by a decrease of remaining cell-surface expressed bait.

The present invention thus also relates to chimeras comprising an amino acid residue sequence containing: 1) a N-terminal signal sequence (SP); 2) a first amino acid tag; 3) a bait sequence for constitutively secreted proteinase cleavage; 4) a second amino acid tag; 5) a transmembrane domain; and 6) either a) a short cytoplasmic signal (short CT) that targets the chimera via the constitutive secretory pathway (ER, Golgi, TGN) to the cellular membrane and have it remain there; or b) a full length CT that allows the chimera once it reaches the cellular membrane to be recycled through early endosomes/lysosomes/acid compartments.

The chimera containing a short CT sequence (e.g. the ACE2 CT ending at FTGIRDR-stop (SEQ ID NO: 5)) is cleaved at the TGN and cell surface since it can no longer be recycled in endosomes. However, chimera containing a full-length CT (e.g. the full length CT of ACE2) including the Y-X-X-hydrophobic motif (Jadot, 1992) (e.g. the Y-A-S-I sequence (SEQ ID NO: 6) present in the full-length CT of ACE2) are sorted from the cellular membrane towards endosomes/lysosomes/acid compartments. This full length-CT containing chimera is desirable for proteinases that process in endosomes/acidic compartments such as MMPs, BACE, NARC-1/PCSK9. Modulators for Furin, PC7, PACE4, PC5B and SKI-1 can be identified with either construct because they are able to process precursors both in endosomes/lysosomes/acid compartments and in other constitutive secretory pathways compartments. In the case of the membrane bound PCs such as Furin, PC5B and SKI-1, processing can also occur at the cell surface.

Assays of the present invention advantageously mimic the environment in which inhibitors will have to work in vivo (i.e. using endogenous proteinases and selecting for cell-diffusible inhibitors effective in the secretory pathway). In specific embodiments, they are advantageously positive assays (i.e., selects for re-appearance of a signal on the cell surface).

The assays of the present invention eliminate molecules that could affect the synthesis or the trafficking of the substrate and those that are toxic to cells. The loss of synthesis or trafficking of the chimera of the present invention to the cell surface will be interpreted as a negative since no HA-Tag will appear at the cell surface.

The present invention provides for the detection of specific proteinase activity through the use of a specific bait domain.

The cell-based assays of the present invention allow for high throughput screening of candidate compounds.

### Cellular Targeting of the Chimeric Protein

The signal peptides (SP) are well-known and direct the synthesis of nascent proteins to the lumen of the endoplasmic reticulum (ER), thus allowing their trafficking towards the rest of the secretory pathway. Additional signals are also required to further determine whether or not a protein will be sorted through a particular secretory pathway including the cytoplasmic membrane. These include trans-membrane domains (TM) as well as signals present within the cytoplasmic tails (CT) of several proteins. For example, signals determining TGN targeting of Furin include amino acids of the cytoplasmic tail. Indeed, two independent targeting signals, which consist of the acidic peptide CPSDSEEDEG₇₈₃ (SEQ ID NO: 7) and the tetrapeptide YKGL₇₆₅ (SEQ ID NO: 8) (an example of Y-X-X-hydrophobic motif) were previously identified that control the recycling of the constitutively secreted Furin from the cell surface to the TGN. The YKGL (SEQ ID NO: 8) is a determinant for targeting from the cell surface to the endosomes, while the acidic peptide signal in the cytoplasmic tail is necessary and sufficient to target Furin from the endosomes to the TGN. The chimeric protein of the present invention carefully combined several sorting signals (SP, TM, CT) to allow for chimeras to either remain at the cell surface and not be reinternalized (short CT) or to be recycled in endosomes (FL-CT). These chimeras were constructed so as to allow their specific targeting to cellular membrane through the ER and TGN via the constitutive secretory pathway. In addition, the sorting signals of the present invention are also combined, in an orderly fashion, with two tag domains sandwiching the bait sequence. The components were carefully chosen to allow proper folding of the chimeric protein in the ER and correct targeting of the mature protein to the cellular membrane from the TGN. The addition of different signals at the C-terminus such as a specific α-helix (as was done for PC1) would change the route of the chimera towards the regulatory pathway and hence target enzymes such as PC1 and PC2 present within dense core granules.

### Two Tags Separated by a Bait Domain: Selection of Cell Clones Optimal for the Cell-Based Assay

The presence of a first tag (i.e. the hemaglutinin A (HA) domain) removed by the activity of a convertase (e.g. Furin), and a second enzyme activity-independent tag (i.e. Fc portion of mouse IgG) allowed the selection by FACS of clones expressing at their cell surface high levels of the chimeric protein completely processed by one or more endogenous PCs (e.g. Furin and PC7). These two characteristics—combining low level of the first tag with the expression of the chimera at the cell surface—makes the selected clones perfectly adapted to the screening of compounds inhibitory for the selected constitutively secreted proteinase (e.g. Furin), or set of constitutively secreted proteinase (e.g., Furin, PC7 etc. . . . ) depending on the choice of the bait sequence. The cleavage of the first tag (i.e. HA) could occur at any step of the secretory pathway including at the cellular membrane and thus could also occur through the activity of enzymes located outside the cell (i.e. MMPs).

### Two Tags Separated by a Bait Domain: Selection of Inhibitory Compounds

In the presence of inhibitory compounds, the cell-surface chimeric protein will harbor the first tag (i.e. the HA domain). Inhibition of the proteinase activity (e.g. the Furin activity) on the bait domain implies that the compound is able to enter the cell and reach the TGN or other compartments of the constitutive pathway without having adverse toxic effects on the cell.

The inhibitory compound could inhibit the catalytic site of the enzyme or other allosteric sites that impact on the productive catalytic activity of the convertase. These compounds can then be tested in vitro to define their exact mechanism of action. However, it is also conceivable that some compounds will act in cellular compartments that control the folding of the convertase, e.g. in the ER, but then this could also affect other proteins and likely lead to cellular stress and death. Such compounds would not be picked up by the cell-based assays of the present invention.

### Selection of Clones

### For Inhibitory Compounds Screening

Recombinant cellular clones optimal for selecting inhibitors in cell-based assays of the present invention express at their cell surface a level as low as possible of a first tag (e.g. HA) and a high level of a second tag (e.g. Fc) that indicates that the chimeric protein was properly expressed and that the bait sequence was cleaved by the subject proteinase. As a result, in such clone, the contrast between a positive (i.e. the candidate compound prevented the subject proteinase from cleaving the bait which resulted in the appearance of a large amount of the first tag at the cell surface) and a negative (i.e. the candidate compound did not prevent the subject proteinase from cleaving the bait which resulted in the appearance of very little or no amount of the first tag at the cell surface) is maximized.

### For Activatory Compounds Screening

Recombinant cellular clones for selecting activators in cell-based assays of the present invention express at their cell surface a level of the first tag (e.g. HA) that is high enough to provide a measurable contrast between a positive and a negative. The level of the second tag (e.g. Fc) should be high enough to show that the clone expresses a sufficient amount of the subject proteinase. Hence the presence of an activatory compound (i.e. the candidate compound promoted cleavage of the bait by the subject proteinase) will result in a decrease of the appearance of the first tag at the surface of the cell.

Alternatively, recombinant cellular clones optimal for selecting activators in cell-based assays of the present invention express at their cell surface a level of the first tag (e.g. alkaline phosphatase) high enough to provide a measurable contrast between a positive and a negative combined to the detection in the culture supernatant (or culture medium) of an amount of the first tag (e.g. alkaline phosphatase) that is low enough to provide a measurable contrast between a positive and a negative but still present to indicate that the chimeric protein was properly expressed. As a consequence, the cellular clones express a level of a second tag at the cell surface (e.g. Fc) that is high enough to indicate that the chimeric protein was properly expressed. As a result, in such clone, the contrast between a positive (i.e. the candidate compound increased the subject proteinase activity on bait cleavage which resulted in the appearance of a large amount of the first tag in the culture supernatant) and a negative (i.e. the candidate compound did not activate the subject proteinase activity on bait cleavage which resulted in the appearance of very little or no amount of the first tag in the culture supernatant) is maximized.

### Host Cells

Although the assays described herein use specific host cells, the present invention should not be so limited. Any cell, preferably human expressing the proteinase that is to be screened for modulators can be used. The use of human cells is preferred for selecting a modulator effective in human.

Hence, for Furin-like PCs any cell expressing Furin-like PCs could be used. Without being so limited, the following cells could be used: HuH7 and HeLA. There are however specific advantages to using HeLA cell lines in Furin-like specific assays of the present invention. They are robust and well-adapted to the high-throughput format, and have been extensively used for HIV work.

For PC5B, any cell expressing PC5B could be used. Without being so limited, the following cells could be used: human lung epithelial A549 cells and mouse adrenal cortex Y1 cells.

For NARC-1/PCSK9, several cell lines expressing NARC-1/PCSK9 could be used. Without being so limited, the following cells could be used: human HuH7, HepG2, CaCo2 as well as LoVo-C5.

In specific embodiments, the cell line preferably overexpresses NARC-1/PCSK9 and presents a low level of LDLR at the cell surface. The HuH7 cell line appears to be one of the best human cell lines to perform the assay, as these cells are of hepatic origin, express endogenously NARC-1/PCSK9 and LDLR, and overexpression of NARC-1/PCSK9 in these cells causes the degradation of the LDLR. However any cell expressing LDLR and NARC-1/PCSK9 or an appropriate mutant thereof (e.g. NARC-1/PCSK9 S127R) could be used in this specific embodiment of the present invention including stable HepG2.

For BACE, several cell lines expressing BACE could be used. Without being so limited, the following cells could be used: mouse Neuro2A, human HuH7, HEK293, HeLa cells, SH-SY5Y neuronal cells, etc.

For SKI-1, several cell lines expressing SKI-1 could be used. Without being so limited, the following cells could be used: human HuH7, HEK293, HepG2, HeLa, etc.

### Detection of the Tag

The detection of the tag may comprise a directly detectable (e.g., a fluorophore) or indirectly detectable (e.g., an enzyme activity allowing detection in the presence of an appropriate substrate) measurement. It could also be measured by the binding of a ligand to the tag (e.g. an antibody or a protein). The detection is preferably performed on intact cell but could also be performed on different fractions of a cell lysate. Under certain circumstances, for instance to monitor an increase of the PC activity, the culture supernatant is used (e.g. increase release of the tag harboring an enzyme activity). The detection step could be monitored by any number of means including, but not limited to, optical spectroscopy, fluorimetry, and radioactive label detection and could use various techniques such as Western blot, Fluorescence Activated Cell Sorting (FACS) and Immuno-Assay performed on intact cells.

Although the present invention is not specifically dependent on the use of a label for the detection of a tag, such a label might be beneficial, by increasing the sensitivity of the detection. Furthermore, it enables automation. Tags can be labeled according to numerous well known methods (Sambrook et al., 1989, supra). Non-limiting examples of labels include ³H, ¹⁴C, ³²P, and ³⁵S. Non-limiting examples of detectable labels include fluorophores, chemiluminescent agents, enzymes, and antibodies including a antibody coupled to ALP (e.g. antibody attached to an alkaline phosphatase such as that illustrated in FIGS. 6 and 15). Other detectable ligands for use with tags, which can enable an increase in sensitivity of the assays of the invention, include biotin and radionucleotides. It will become evident to the person of ordinary skill that the choice of a particular label dictates the manner in which it is bound to the tag. As used herein the term “ligand” to the first or the second amino acid tag according to specific embodiments of the present invention thus refer to any ligand able to bind the first or the second amino acid tag. Without being so limited, when the tag is HA, the ligand may be any anti-HA monoclonal or polyclonal antibody.

More specifically, in accordance with an aspect of the present invention, there is provided a chimeric protein comprising in sequence a signal peptide, a first amino acid tag, a proteinase bait, a second amino acid tag, a transmembrane domain and a cytosolic domain, wherein the cytosolic (CT) domain comprises a sequence able to recycle the protein from the cellular membrane to endosomes. In a specific embodiment of the chimeric protein, the second tag is an immunoglobulin Fc fragment of at least 20 amino acid residues. In an other specific embodiment, the CT comprises a Y-X-X-hydrophobic motif, wherein X is any amino acid. In an other specific embodiment, the CT domain is the full-length CT of ACE2 as set forth in SEQ ID NO: 73. In an other specific embodiment, the first tag is a hemaglutinin A domain (HA). In an other specific embodiment, the bait has a length of 9 to 30 amino acid residues.

In an other specific embodiment, the bait comprises an amino acid sequence as set forth in the formula (K/R)-(X)n-(K/R), where n=0 (SEQ ID NO: 1), 2 (SEQ ID NO: 2), 4 (SEQ ID NO: 3) or 6 (SEQ ID NO: 4) and X is any amino acid. In an other specific embodiment, the bait comprises an amino acid sequence as set forth in KRIRLRRSPD (SEQ ID NO: 29). According to another specific embodiment, there is provided a chimeric protein, the amino acid sequence of which is as set forth in SEQ ID NO: 48. In an other specific embodiment, the chimeric protein is encoded by a nucleotide sequence as set forth in SEQ ID NO: 47.

In an other specific embodiment, the bait comprises KRIRLRRLPD (SEQ ID NO: 76).

In an other specific embodiment of the chimeric protein, the bait comprises an amino acid sequence as set forth in the formula R-X-(L/V)-Z, wherein X is any amino acid and Z is any amino acid except E, D, C, P and V (SEQ ID NO: 9). In an other specific embodiment, the bait comprises an amino acid sequence as set forth in IYISRRLLGTFS (SEQ ID NO: 30). According to another specific embodiment, there is provided a chimeric protein, the amino acid sequence of which is as set forth in SEQ ID NO: 52. In an other specific embodiment, the chimeric protein is encoded by a nucleotide sequence as set forth SEQ ID NO: 51.

In an other specific embodiment of the chimeric protein, the bait comprises an amino acid sequence as set forth in VFAQSIP (SEQ ID NO: 10). In an other specific embodiment, the bait comprises an amino acid sequence as set forth in SSVFAQSIPWN (SEQ ID NO: 31). In an other specific embodiment, the bait comprises an amino acid sequence as set forth in KHQKLLSIDLD (SEQ ID NO: 32). According to another specific embodiment, there is provided a chimeric protein, the amino acid sequence of which is as set forth in SEQ ID NO: 57. In an other specific embodiment, the chimeric protein is encoded by a nucleotide sequence as set forth in SEQ ID NO: 56. According to another specific embodiment, there is provided a chimeric protein, the amino acid sequence of which is as set forth in SEQ ID NO: 59. In an other specific embodiment, the chimeric protein is encoded by a nucleotide sequence as set forth in SEQ ID NO: 58.

In an other specific embodiment of the chimeric protein, the bait comprises an amino acid sequence as set forth in KISEVNLDAE (SEQ ID NO: 33). In an other specific embodiment, the bait comprises an amino acid sequence as set forth in KISEVNFEVE (SEQ ID NO: 34). According to another specific embodiment, there is provided a chimeric protein, the amino acid sequence of which is as set forth in SEQ ID NO: 63. In an other specific embodiment, the chimeric protein is encoded by a nucleotide sequence as set forth in SEQ ID NO: 62. According to another specific embodiment, there is provided a chimeric protein, the amino acid sequence of which is as set forth in SEQ ID NO: 67. In another specific embodiment, the chimeric protein is encoded by a nucleotide sequence as set forth in SEQ ID NO: 66.

In accordance with another aspect of the present invention, there is provided a cell line stably expressing the chimeric protein of the present invention and expressing a proteinase able to cleave the bait of the chimeric protein. In another specific embodiment of the cell line, the cell line is a HeLa cell line. In another specific embodiment, the cell line is a human lung epithelial A549 cell line. In another specific embodiment, the cell line is a HuH7 cell line. In another specific embodiment, the cell line is a HuH7 cell line. In another specific embodiment, the cell line overexpresses NARC1/PCSK9 or the S127R mutated form of the NARC-1/PCSK9; and expresses a low level of LDLR at the cell surface. In another specific embodiment, the cell line is a Neuro2A cell line.

In accordance with another aspect of the present invention, there is provided a cell-based assay for identifying a constitutively secreted proteinase modulator, which comprises the steps of: (a) providing a cell line of the present invention; (b) measuring the presence of the first amino acid tag at the cell surface in the presence of a candidate modulator and in the absence thereof, whereby a difference in the level of detection of the tag in the presence of the candidate modulator as compared to in the absence thereof is an indication that the candidate is a constitutively secreted proteinase modulator. In a specific embodiment, the assay is for identifying a constitutively secreted proteinase inhibitor, and the candidate modulator is a candidate inhibitor whereby a higher level of detection of the first amino acid tag in the presence of the candidate inhibitor as compared to in the absence thereof is an indication that the candidate is a constitutively secreted proteinase inhibitor. In another specific embodiment, the assay is for identifying a constitutively secreted proteinase activator, wherein the candidate proteinase modulator is a candidate proteinase activator and wherein the cell line expresses a ratio of first amino acid tag:second amino acid tag between about 10:90 and about 30:70, whereby a lower level of detection of the first amino acid tag in the presence of the candidate activator as compared to in the absence thereof is an indication that the candidate is a constitutively secreted proteinase activator. In another specific embodiment, the assay is performed on an intact cell. In another specific embodiment, the assay is performed using a cell lysate.

In accordance with another aspect of the present invention, there is provided a cell-based assay for identifying a constitutively secreted proteinase modulator, which comprises the steps of: (a) providing a cell line of the present invention; (b) measuring the presence of the first amino acid tag in the cell culture supernatant in the presence of a candidate modulator and in the absence thereof, whereby a difference in the level of detection of the tag in the presence of the candidate modulator as compared to in the absence thereof is an indication that the candidate is a constitutively secreted proteinase modulator. In an specific embodiment, the assay is for identifying a constitutively secreted proteinase activator, wherein the candidate proteinase modulator is a candidate proteinase activator whereby a higher level of detection of the first amino acid tag in the supernatant in the presence of the candidate activator as compared to in the absence thereof is an indication that the candidate is a constitutively secreted proteinase activator.

In other specific embodiments of the assays of the present invention, the presence of the first amino acid tag is directly measurable using fluorometry. In other specific embodiments the presence of the first amino acid tag is measurable through measurement of the activity an enzyme on a substrate. In a more specific embodiment, the enzyme is alkaline phosphatase. In another specific embodiment, the presence of the first amino acid tag is measurable by the binding of a ligand to the first amino acid tag.

In accordance with another aspect of the present invention, there is provided a SKI-1 convertase substrate as set forth in Succ-YISRRLL-MCA (SEQ ID NO: 36).

In accordance with another aspect of the present invention, there is provided a SKI-1 convertase inhibitor as set forth in dec-YISRRLL-cmk (SEQ ID NO: 42).

In accordance with another aspect of the present invention, there is provided a SKI-1 convertase inhibitor as set forth in dec-ISRRLL-cmk (SEQ ID NO: 43).

In accordance with another aspect of the present invention, there is provided a SKI-1 convertase substrate as set forth in Succ-ISRRLL-MCA (SEQ ID NO: 37).

In accordance with another aspect of the present invention, there is provided a use of the inhibitor of the present invention in the preparation of a medicament.

As used herein the term “proteinase” refers to an enzyme that breaks down proteins into their component peptides. Without being so limited, it includes metalloprotease (including MT-MMP and ADAM), aspartyl proteinases such as BACE, cysteine proteinases, serine proteinases including PCs, and threonine proteinases.

As used herein, the term “Furin-like PCs” or “AA-specific PCs” is meant to refer to any member of the dibasic-specific amino acid specific PCs, including PC5/PC6, Furin, PACE4, PC4, PC7/PC8, PC1/PC3 and PC2.

As used herein, the term “proteinase modulator” refers to a proteinase inhibitor or to a proteinase activator. It includes proteins, peptides and small molecules.

As used herein the term “PC-like” refers to all PCs constitutively secreted such as Furin, PC5, PACE4, PC4, PC7, SKI-1 and NARC-1/PCSK9 (see FIG. 4).

The present invention thus relates to chimeras comprising an amino acid residue sequence containing: 1) a N-terminal signal sequence (SP); 2) a first amino acid tag; 3) a bait sequence for constitutively secreted proteinase cleavage; 4) a second amino acid tag; 5) a transmembrane domain; and 6) either a) a short cytoplasmic signal (short CT) that targets the chimera via the constitutive secretory pathway (ER, Golgi, TGN) to the cellular membrane and have it remain there; or b) a full length CT that allows the chimera once it reaches the cellular membrane to be recycled through early endosomes/lysosomes/acid compartments.

### N-Terminal Signal Sequence

Proteins destined for export, for location in a membrane and more generally for the secretory pathway contain a signal peptide comprising the first 20 or so amino acids at the N-terminal end and always includes a substantial number of hydrophobic amino acids. Several peptide signals are known and could be used in the present invention. For instance, SPdb, a signal peptide database lists a number of useful signal peptides (Choo K H, Tan T W, Ranganathan S. 2005. SPdb—a signal peptide database. BMC Bioinformatics 6:249). Without being so limited, useful signal peptides include those of human insulin, renin as well as those of PCs themselves amongst others.

### Amino Acids Tags

The first amino acid tag could be any sequence detectable by an appropriate antibody or binding protein. Without being so limited, they include hemaglutinin A (HA), c-myc tag, V5 (Invitrogen). The first amino acid tag could also be a fluorescent amino acid sequence (i.e. a green fluorescent protein) or a sequence associated with a detectable enzymatic activity (i.e. alkaline phosphatase).

The second amino acid tag could be any sequence detectable by an appropriate antibody or other ligand as well as any fluorescent amino acid sequence distinct from the first tag used. The second tag is ideally longer than the first tag, preferably a segment longer than 20 amino acids, that correctly folds in the ER and for which a detection system exists using an antibody or a binding protein (e.g. GST). The choice of the Fc as second tag for specific embodiments of the present invention was carefully made in view of data showing that this domain could fold on its own (Jutras et al., 2000). Without being so limited, in specific embodiments of the present invention, second tags include human, mouse or other animal immunoglobulin Fc fragments.

### Bait Sequence

The baits used in the chimeras of the present invention may be any sequence that is known to be cleavable by the proteinase targeted by the assay. Desirably, the sequence is specific to that proteinase. For SKI-1/PCSK8, any sequence comprising a sequence satisfying the formula in FIG. 1 or comprising any of the sequences disclosed in FIG. 3 for instance is appropriate. For BACE, a sequence comprising one of those disclosed in FIG. 5 for instance is appropriate. For the PC-like proteinases, any sequence comprising a sequence satisfying the formula disclosed in FIG. 1 for the basic-aa-specific PCs for instance is appropriate. For NARC-1/PCSK9, any sequence comprising the sequence disclosed in FIG. 1 or FIG. 5 for instance is appropriate. For MMPs, the teaching of Turk, B. E. et al (2001) on how to determine proteinase site motifs using mixture-based oriented peptide libraries and disclosing certain MMPs preferences is relevant. The baits are generally of a size between 9 and 30 amino acids.

### Transmembrane Domain

Proteins destined for location in the membrane contain a transmembrane domain comprising a stretch of 15 to 22 hydrophobic amino acids in an alpha helical secondary conformation. Several transmembrane domains are described and could be used in the present invention. TMbase™ is a database of transmembrane proteins (Hofmann K. and Stoffel W. 1993. TMBASE—A database of membrane spanning protein segments Biol. Chem. Hoppe-Seyler 374, 166) with their helical membrane-spanning (TM) or (M) domain. Without being so limited, they include a TM derived from the human angiotensin converting enzyme-2 (ACE2 i.e. the SARS-Corona Virus receptor).

### Cytoplasmic Signal

Convenient cytoplasmic signals for use in the chimeras of the present invention include any signal that targets the chimera from the cellular membrane to the endosomes/lysosomes/acid compartments through the recycling pathway. The present invention encompasses two types of CT-signals: a short one (short CT 3-8 aa) that limits the trafficking up to the cell surface and can no longer internalize in clathrin coated endosomes; and a long one (FL-CT up to 50 aa) that has specific internalization signals (e.g., Y-X-X-hydrophobic) that can recycle to the endosomes/lysosomes and if associated with an acidic motif, for example, will recycle to the TGN. Without being so limited, long CTs include the cytoplasmic tail of ACE2, that of the LDL receptor, or that of Furin, all of which cycle from the cellular membrane to the endosomes and TGN and via the constitutive secretory pathway.

Since many ligands are antibodies, a particular sub-class of assays of the present invention will be referred to as CELISA assays (CELI-based ImmunoaSsay and Activity).

Other objects, advantages and features of the present invention will become more apparent upon reading of the following non-restrictive description of specific embodiments thereof, given by way of example only with reference to the accompanying drawings.

### BRIEF DESCRIPTION OF THE DRAWINGS

In the appended drawings:

FIG. 1 presents motifs recognized by the various PCs where X is any amino acid residue except Cys and Z is any amino acid except E, D, C, P and V. The motif for dibasic-specific amino acid PCs is shown (SEQ ID NOs: 1-4); along with one for SKI-1 (SEQ ID NO: 9), and one for NARC-1/PCSK9 (SEQ ID NO: 10);

FIG. 2 presents the reactive site loop (RSL) domain and bait region of human α1-AT (SEQ ID NO: 11), α1-PDX (SEQ ID NO: 12), and Spn4.1 (SEQ ID NO: 13). The arrow indicates the cleavage site for the PCs;

FIG. 3 presents various SKI-1 substrates from mammalian and viral precursors and their cleavage sites shown with an arrow (SEQ ID NOs: 14 to 28);

FIG. 4 schematically presents the cell localizations where various PCs cleave their substrates in the secretory and endocytic pathways. ER: endoplasmic reticulum; TGN: trans Golgi network; SG: secretory granule; S: Serine residue from the active site from the PC-like; Endo: endosome; Prosegment; PC-derived inhibitory prosegment;

FIG. 5 schematically presents the structure of chimeras of the present invention and specific bait sequences for: constitutively secreted Furin-like PCs (SEQ ID NO: 29), SKI-1/PCSK8 (SEQ ID NO: 30), NARC-1/PCSK9 (SEQ ID NOs: 31 and 32), BACE-APP swedish (sw) (SEQ ID NO: 33), and BACE-APP Swedish mutant (mut) (SEQ ID NO: 34); SP stands for signal peptide; HA stands for hemaglutinin A tag; M stands for transmembrane; CT stand for cytosolic tail; and ACE2 for angiotensin converting enzyme 2;

FIG. 6 schematically presents the mechanism of a CELISA of the present invention;

FIG. 7 graphically shows the detection of HA tags on the surface of a stably transfected pool of HeLa cells as measured with: (A) a fluorescence-activated cell sorting (FACS) before (darker dots) and after (paler dots) application of 60 μM of the convertase peptidic inhibitor dec-RVKR-cmk (SEQ ID NO: 35); and (B) a CELISA assay in the presence of either 15, 30 or 60 μM dec-RVKR-cmk (SEQ ID NO: 35);

FIG. 8 graphically shows the detection of HA tags on the surface of a single clone, PCfur-1.6, derived from the HeLa cells described in FIG. 7. This clone was selected by FACS to express high Fc but low HA immunoreactivity at the cell surface. (A) FACs analysis for the HA tag in untreated (left panel) and treated (right panel) HeLa PCfur-1.6 clone with 30 μM dec-RVKR-cmk (SEQ ID NO: 35). (B) Effect of inhibition of Furin-like PCs by the serpin α1-PDX (as compared to the non-inhibitory α1-antitrypsin, α1-AT) on the CELISA assay. The HA tag is measured on a PCfur-1.6 clone transfected with cDNAs coding for these serpins (Transfection) or infected using recombinant adenoviruses (Infection). The numbers above the bars represent the fold increase in the detection of the HA tag signal in the presence of α1-PDX versus α1-AT. (C) Western blot analysis of the same cells infected with adenovirus expressing either α1-AT (AT) or α1-PDX (PDX) at two mutiplicities of infection (1×=1×10⁸ and 2×=2×10⁸ infectious adenoviral particles). Western horseraddish peroxidase coupled streptavidin (WB streptavidin-HRP). The bands at 50 kDa and 35 kDa are non specific;

FIG. 9 graphically shows the optimization of a CELISA assay. (A) optimization of cell density, where the number of cells/well is optimal between 15,000-5,000 cells/well; (B) optimization of the HA antibody incubation time period for a 7,500 cells/well assay. It shows that incubations of 4-8 h are optimal. (C) optimization of the number of washes following the antibody incubation period. A minimum of 4 washes is recommended. CMK: dec-RVKR-cmk (SEQ ID NO: 35);

FIG. 10 shows a summary of the SKI-1 activity on MCA-conjugated various viral glycoprotein peptide substrates (I: SEQ ID NO: 36; II: SEQ ID NO: 37; III: SEQ ID NO: 38; IV: SEQ ID NO: 39; V: SEQ ID NO: 40; VI: SEQ ID NO: 41) +++: much better cleavage than +; −: no cleavage. The viral recognition site motifs were derived from Lassa (LAV), Crimean Congo hemorrhagic fever (CCHFV) and Lymphocytic Choriomeningitis (LCMV);

FIG. 11 shows the inhibitory effect of dec-YISRRLL-cmk (SEQ ID NO: 42) and dec-ISRRLL-cmk (SEQ ID NO: 43) on the endogenous proSREBP-2 ex vivo processing. CHOK1 cells were treated with medium containing delipidated serum (LPDS), 50 μM compactin and 50 μM sodium mevalonate in the absence or presence of different concentrations of (A) dec-YISRRLL-cmk (SEQ ID NO: 42) or (B) dec-ISRRLL-cmk (SEQ ID NO: 43) for 18 h. Western blot analyses of the cell lysates were performed using a mouse monoclonal antibody directed against the NH₂-terminal domain of hamster SREBP-2. The arrows point to the migration position of the precursor proSREBP-2 and its mature nuclear form nSREBP-2;

FIG. 12 shows the inhibitory effect of dec-YISRRLL-cmk (SEQ ID NO: 43) and dec-RRLL-cmk (SEQ ID NO: 44) on the endogenous proATF6 ex vivo processing. Following transient transfection of CHOK1 cells with a cDNA coding for ATF6-Flag, the cells were treated with varying concentrations of (A) dec-RRLL-cmk (SEQ ID NO: 44) or (B) dec-YISRRLL-cmk (SEQ ID NO: 43) in the presence of 2 μg/ml tunicamycin for 12 h. Western blot analyses of the cell lysates were performed using an anti-FLAG M2 monoclonal antibody. The arrows point to the migration position of the precursor proATF6 and its mature nuclear form nATF6;

FIG. 13 shows that dec-YISRRLL-cmk (SEQ ID NO: 42) is not an effective inhibitor of the ex vivo processing of propDGF-A. On day 1, HEK293 cells, stably expressing PDGF-A-V5 construct, were incubated overnight in serum free medium with varying concentrations of (A) dec-YISRRLL-cmk (SEQ ID NO: 42) or (B) dec-RVKR-cmk (SEQ ID NO: 35). On day 3, the media were fractionated on 12% SDS-PAGE and then analyzed by Western blot using a V5-HRP antibody. The arrows point to the migration position of the precursor propDGF-A and its mature form PDGF-A;

FIG. 14 shows the Western blot analysis obtained from cells expressing chimera containing the following NARC-1/PCSK9 bait sequence: SSVFAQ-SIPWN (SEQ ID NO: 31) (SN11). Cells were treated or not for 6 h with 50 μM of dec-RVKR-cmk (SEQ ID NO: 35) used herein as an activator of NARC-1/PCSK9. Dec-RVKR-cmk (SEQ ID NO: 35) being an inhibitor of Furin-like enzymes and furin being an inhibitor of NARC-1/PCSK9 (see Pasquato et al, 2006). Dec-RVKR-cmk (SEQ ID NO: 35) is thus an activator of NARC-1/PCSK9. Cells express an approximately equal amounts of ER- (endo H sensitive, not shown) and Golgi-associated (endo H resistant, not shown) SN11-containing chimera as detected using an HA mAb. In the presence of dec-RVKR-CMK (SEQ ID NO: 35) a substantial decrease in the level of the Golgi-associated HA versus the ER-one is observed. As a control, the level of the Fc immunoreactivity in presence of dec-RVKR-cmk (SEQ ID NO: 35) was also measured and showed that the Golgi form that lost most of its HA tag, is still very positive for Fc;

FIG. 15 schematically shows a NARC-1/PCSK9-LDLR coupled CELISA detection assay for the identification of compounds causing the NARC-1/PCSK9 inhibition along with the subsequent accumulation of LDLR at the cell surface;

FIG. 16 shows an example of a high throughput screening assay specific for CELISA-Furin-like PCs. A total of 15,000 compounds were tested. Each dot represents the percentage of intensity of the blue green reaction product read at 405 nm by a plate reader spectrometer. This measure is indicative of the presence of HA at the cell surface, as expressed relative to the intensity obtained in the presence of positive inhibitory dec-RVKR-cmk (SEQ ID NO: 35) controls spread through the plate reader every 200 wells. Two positive hits are indicated;

FIG. 17 shows the cDNA nucleotide sequence (SEQ ID NO: 45) and the amino acid sequence (SEQ ID NO: 46) of a chimeric protein comprising bait KRIRLRRSPD (SEQ ID NO: 29) for constitutively secreted Furin-like PCs and a short ACE2 CT;

FIG. 18 shows the cDNA nucleotide sequence (SEQ ID NO: 47) and the amino acid sequence (SEQ ID NO: 48) of a chimeric protein comprising bait KRIRLRRSPD (SEQ ID NO: 29) for constitutively secreted Furin-like PCs and a full-length ACE2 CT;

FIG. 19 shows the cDNA nucleotide sequence (SEQ ID NO: 49) and the amino acid sequence (SEQ ID NO: 50) of a chimeric protein comprising bait IYISRRLLGTFS (SEQ ID NO: 30) for SKI-1/PCSK8 and a short ACE2 CT;

FIG. 20 shows the cDNA nucleotide sequence (SEQ ID NO: 51) and the amino acid sequence (SEQ ID NO: 52) of a chimeric protein comprising bait IYISRRLLGTFS (SEQ ID NO: 30) for SKI-1/PCSK8 and a full-length ACE2 CT;

FIG. 21 shows the cDNA nucleotide sequence (SEQ ID NO: 53) and the amino acid sequence (SEQ ID NO: 54) of a chimeric protein comprising bait EEDSSVAQSIPWN (SEQ ID NO: 55) for NARC-1/PCSK9 and a short ACE2 CT;

FIG. 22 shows the cDNA nucleotide sequence (SEQ ID NO: 56) and the amino acid sequence (SEQ ID NO: 57) of a chimeric protein comprising bait SSVAQSIPWN (SEQ ID NO: 31) for NARC-1/PCSK9 and a full-length ACE2 CT;

FIG. 23 shows the cDNA nucleotide sequence (SEQ ID NO: 58) and the amino acid sequence (SEQ ID NO: 59) of a chimeric protein comprising bait KHQKLLSIDLD (SEQ ID NO: 32) for NARC-1/PCSK9 and a full-length ACE2 CT;

FIG. 24 shows the cDNA nucleotide sequence (SEQ ID NO: 60) and the amino acid sequence (SEQ ID NO: 61) of a chimeric protein comprising bait KISEVNLDAE (SEQ ID NO: 33) for BACE-APP sw and a short ACE2 CT;

FIG. 25 shows the cDNA nucleotide sequence (SEQ ID NO: 62) and the amino acid sequence (SEQ ID NO: 63) of a chimera protein comprising bait KISEVNLDAE (SEQ ID NO: 33) for BACE-APP sw and a full-length ACE2 CT;

FIG. 26 shows the cDNA nucleotide sequence (SEQ ID NO: 64) and the amino acid sequence (SEQ ID NO: 65) of a chimeric protein comprising bait KISEVNLEVE (SEQ ID NO: 34) for BACE-APP sw (mut) and a short ACE2 CT;

FIG. 27 shows the cDNA nucleotide sequence (SEQ ID NO: 66) and the amino acid sequence (SEQ ID NO: 67) of a chimeric protein comprising bait KISEVNLEVE (SEQ ID NO: 34) for BACE-APP sw (mut) and a full-length ACE2 CT; and

FIG. 28 details the amino acid sequences of chimeric proteins presented in FIGS. 17 to 27 (Panel A: SEQ ID NO: 46; panel B: SEQ ID NO: 48; panel C: SEQ ID NO: 50; panel D: SEQ ID NO: 52; panel E: SEQ ID NO: 54; panel F: SEQ ID NO: 57; panel G: SEQ ID NO: 59; panel H: SEQ ID NO: 61; panel I: SEQ ID NO: 63; panel J: SEQ ID NO: 65; and panel K: SEQ ID NO: 67). It shows the localization of the signal peptide (boxed) (SEQ ID NO: 68); of the first tag (bold and italic) (SEQ ID NO: 69); of the bait (bold); of the second tag (italic) (SEQ ID NO: 70); of the transmembrane domain (boxed and bold) (SEQ ID NO: 71); and of the short (SEQ ID NO: 72) or full-length (SEQ ID NO: 73) cytoplasmic tail (underscored).

### DESCRIPTION OF ILLUSTRATIVE EMBODIMENTS

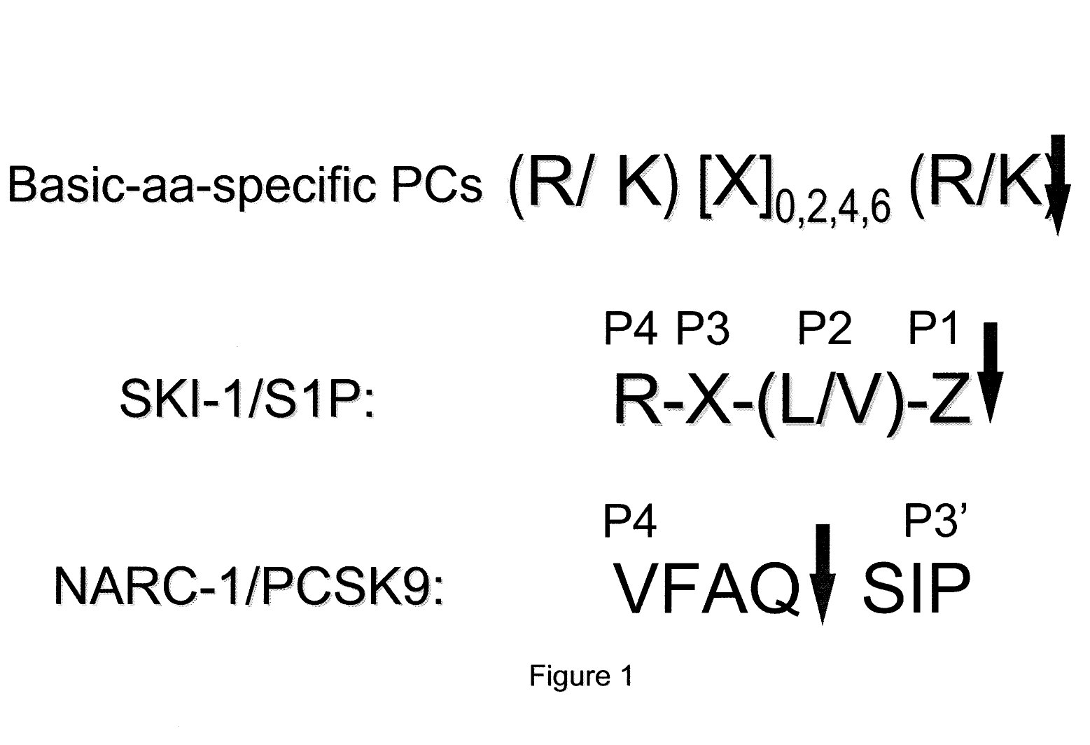

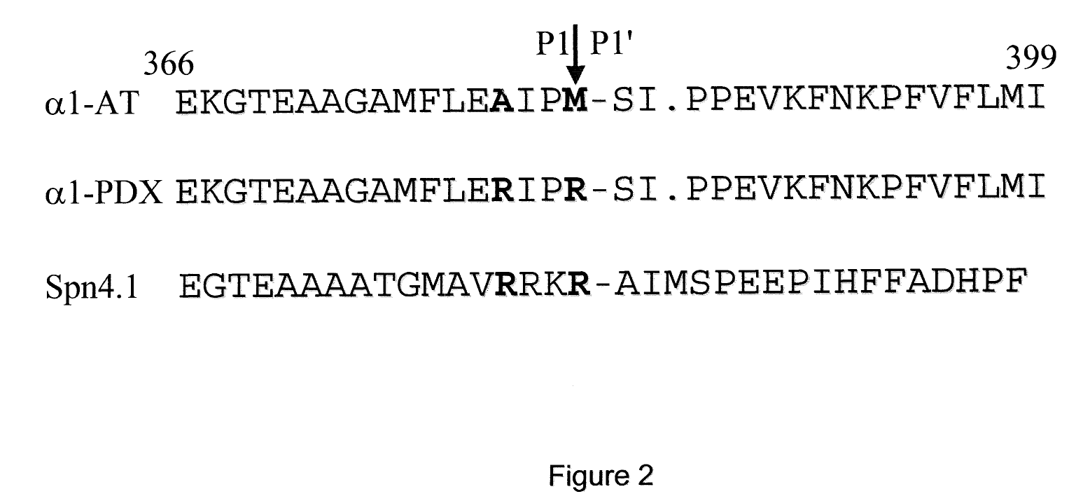

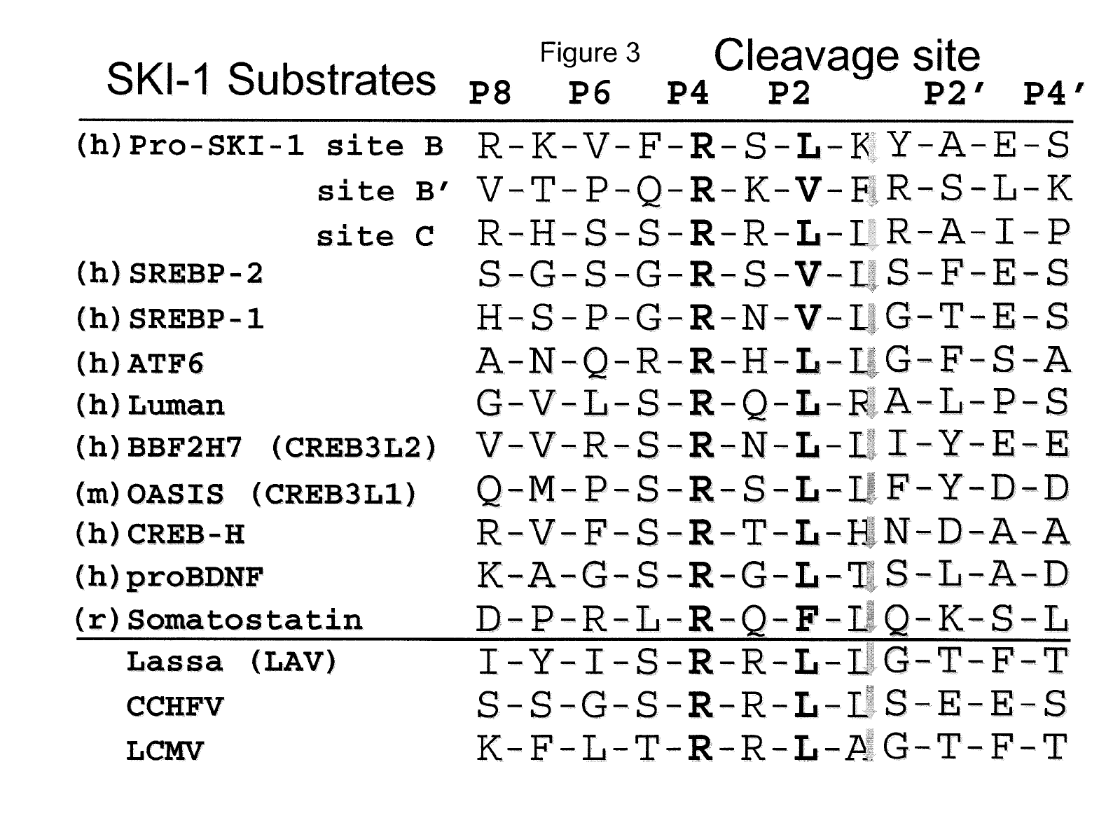

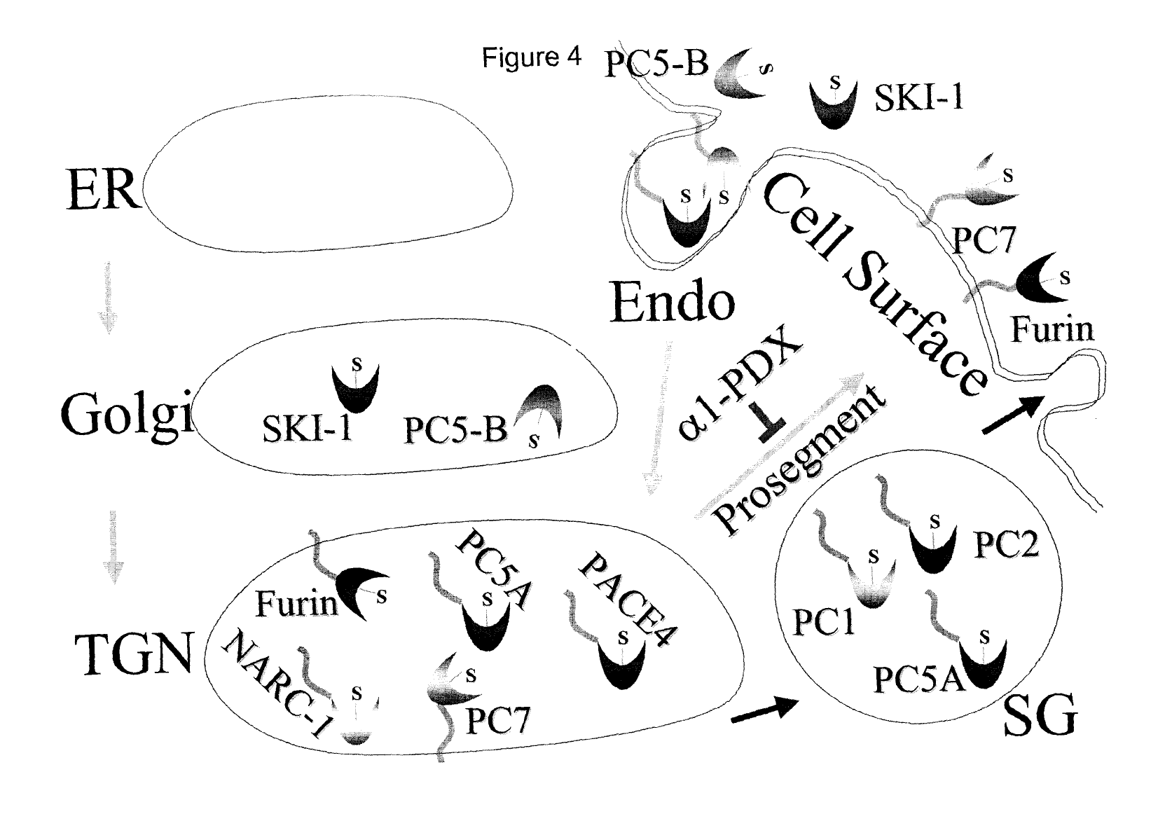

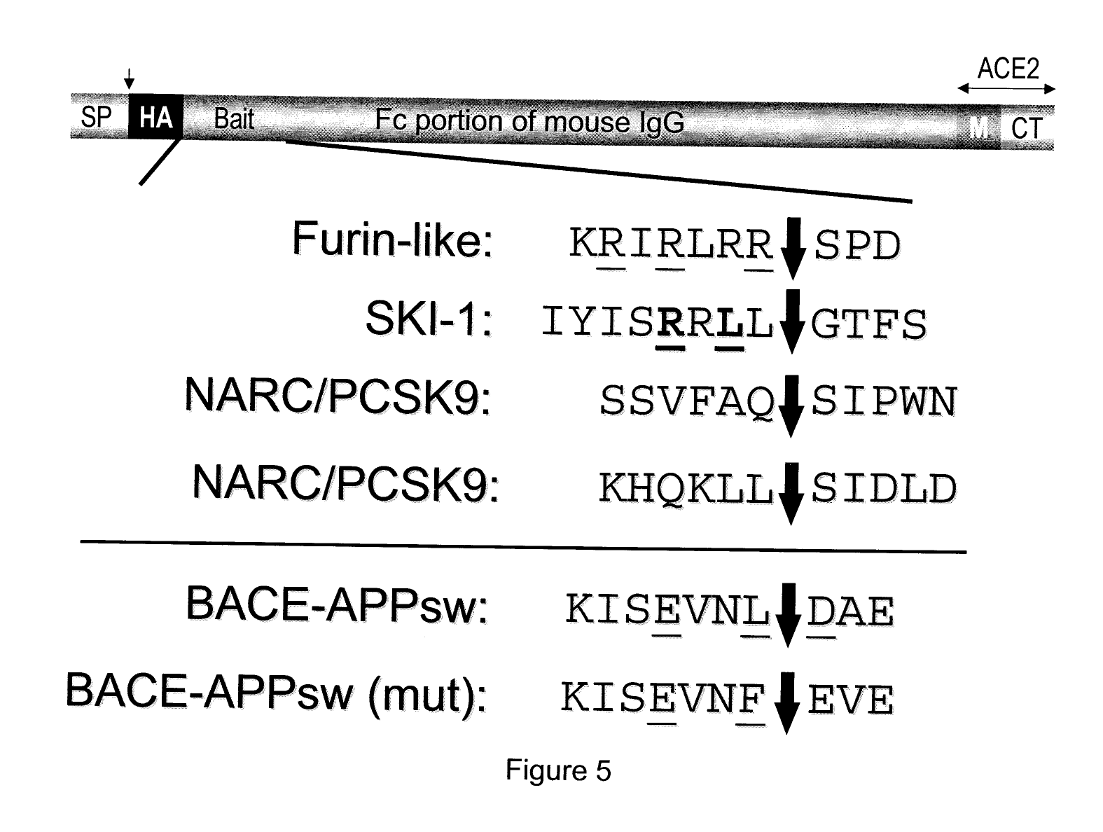

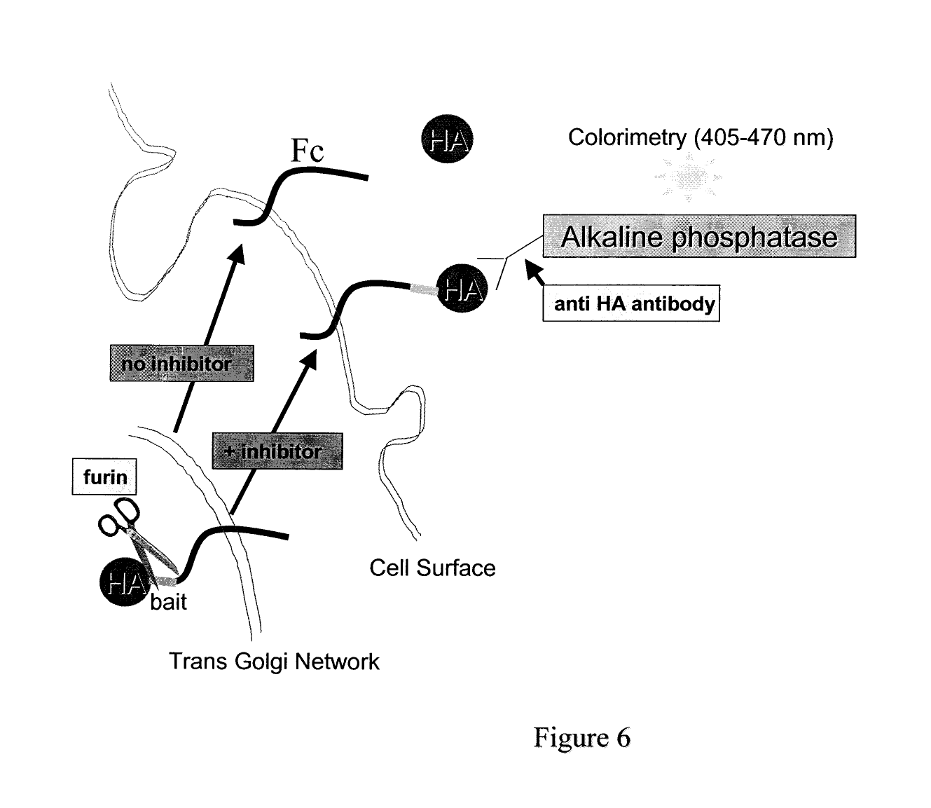

In a first aspect of the present invention, the examples described herein present cells expressing chimeras, each presenting a bait sequence developed in light of knowledge accumulated on the PCs cleavage specificity (e.g. FIGS. 1 and 3), known serpin-protein inhibitors (FIG. 2), prosegment inhibitors such as Spn4.1 (FIG. 2) as well as the cellular biology of the targeted PCs (FIG. 4) and aspartyl proteinase BACE (Data not shown). Thus, the following chimeras were devised 1) for constitutively secreted Furin-like PCs (PC5, PC7, Furin and PACE4); 2) for SKI-1/PCSK8; 3) for NARC-1/PCSK9; 4) for BACE-APP Swedish; and 5) for BACE-APPsw (mut) (FIG. 5). The method chosen is based on positive selection for inhibitors that enhance the cell surface expression of a tagged protein containing a specific bait cleavage motif.

In a second aspect of the present invention, the examples described herein present assays for the identification of NARC-1/PCSK9 inhibitors that induce the reappearance of LDL receptor at the surface of cell.

In a third aspect of the present invention, the examples described herein present new SKI-1 inhibitors.

The present invention is illustrated in further details by the following non-limiting examples presenting sensitive tailor-made cell-based assays designed to isolate PCs inhibitors.

### Example 1

### Cell-Based Inhibitors Screening for Constitutively Secreted Furin-Like PCs

### Construction of Chimera

A chimeric type-I membrane bound cell-surface protein was devised that exhibited the best substrate consensus for Furin, PC5, PC7 and PACE4. Because the HeLa cell, which does not express PC5, was used as host, it could not be screened for PC5 inhibitors.

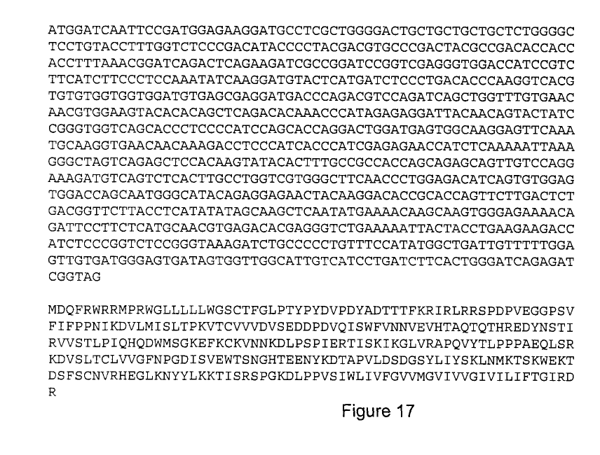

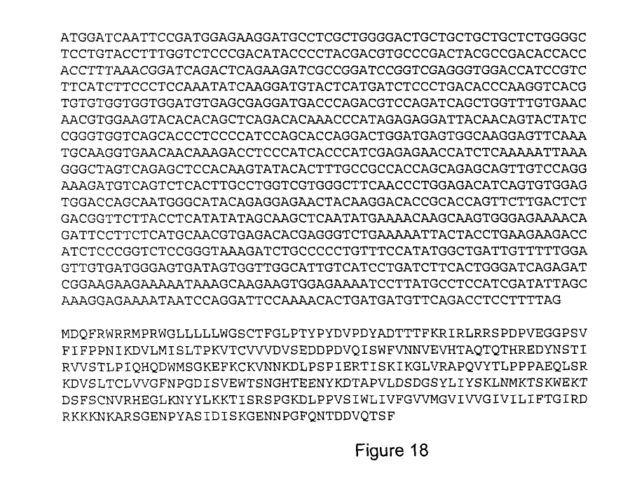

The constructions were obtained by standard PCR and cloning techniques (Wiley, J. & Sons) and was made in the model vector pcDNA3 (Invitrogen). The cDNA and amino acid sequences appear in FIG. 17-18. The chimera presented contained the short ACE2-CT form (FIG. 17) or the full length ACE2-CT form (FIG. 18).

One chimera (SEQ ID NO: 48) obtained consisted of 1) a N-terminal human Renin signal sequence (SP) (SEQ ID NO: 68); followed by 2) a 9 amino acid HA-Tag in bold (YPYDVPDYADTTTF) (SEQ ID NO: 74), where DTTTF (SEQ ID NO: 75) is a linker of HA to the bait sequence, 3) a bait sequence for proteinase cleavage (KRIRLRR-SPD (SEQ ID NO: 29)) followed by 4) a Fc fragment of mouse immunoglobulin in italic (SEQ ID NO: 70); 5) a transmembrane domain (SEQ ID NO: 71); and 6) a full length segment of the cytosolic tail derived from the human angiotensin converting enzyme-2 (ACE2 i.e. the SARS-Corona Virus receptor) (SEQ ID NO: 73). The linker is dispensable and is the result of the specific cloning technique used. The amino acids between the bait and the fragment of mouse immunoglobulin and between the fragment of mouse immunoglobulin and the transmembrane domain (e.g. PV, DPV) are also the result of the specific cloning sequence used.

A bait that is specific for PC5 is made by substituting leucine for serine in the bait sequence as follows: KRIRLRR-LPD (SEQ ID NO: 76). Human lung epithelial A549 cells and mouse adrenal cortex Y1 cells could be used in the cell-based assays of the present invention for identifying PC5 modulators.

Transfection of Hela Cells with Furin-Like PCs Specific Chimera

HeLa cells were transfected with a vector containing the chimera using lipofectamine as described by the manufacturer (Invitrogen). See also (Wiley, J. & Sons).

### Selection of Pools of Cells

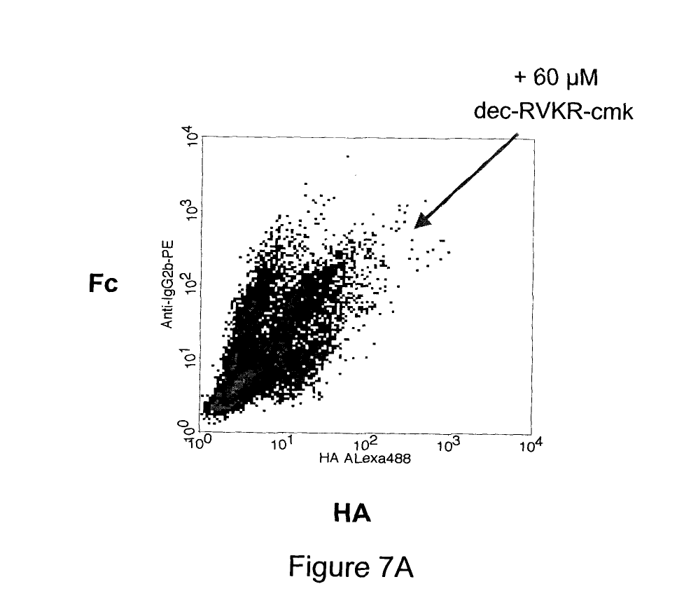

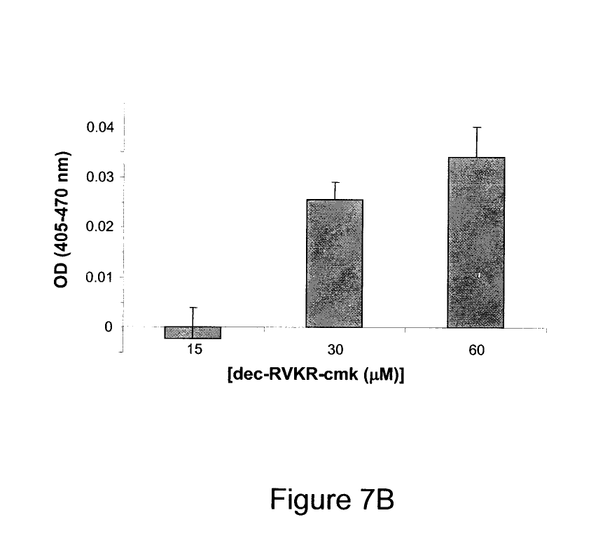

HeLa cells were submitted to two rounds of fluorescence activated cell sorting (FACS) using Alexa488™ as fluorophore with a MoFlo™ cell sorter (Cytomation, Fort Collins, Colo., USA) to obtain pools of cells expressing the Fc (Fc positive) but negative for the HA tag (HA negative) (FIG. 7A, darker dots). The presence of Fc tags was the sign that cleavage by Furin-like PCs at the bait sequence KRIRLRR←SPD (SEQ ID NO: 29) had released the HA tag.

### Detection of Effect of Convertase Inhibitor on HA Tag Appearance on Cell Surface

This isolated pool of cells was incubated for 6 h with 60 μM of dec-RVKR-cmk (SEQ ID NO: 35), a potent PC-inhibitor, and submitted to FACS analysis. A very significant reversion of the level of the chimeric protein to that of the uncleaved form (i.e., re-appearance of the HA tag at the cell surface) was observed (FIG. 7A, paler dots).

A CELISA test was then used to confirm the results obtained by the FACS analysis (FIG. 7A). Pools of cells transfected with the chimera were rinsed twice with PBS. Then, at room temperature, the following steps were performed. Cells were fixed in PBS and 4% formalin 10 min. They were then rinsed twice with PBS. The anti-HA/peroxidase antibody diluted 1/8000 in PBS and 1% powdered milk (filtered) was then added. The cells were incubated 30 minutes on Labquake™. Again, the cells were rinsed twice with PBS. The substrate (2,2′ azino-Bis (3-ethylobenzathiazoline-6-sulfonic acid); Sigma) was added to the cells and the mixture was incubated 15 minutes. The OD was read at 405 nm and 470 nm for turbidity, and the subtraction of the reading at both wavelengths (405-470) gave normalized product absorbance. The CELISA assay was performed in the presence of different concentrations of dec-RVKR-cmk (SEQ ID NO:35). The signal/noise ratio obtained in the presence of 60 μM of dec-RVKR-CMK (SEQ ID NO: 35) was above 15. The CELISA (FIG. 7B) confirmed that the presence of the inhibitor increases the detection of HA at the surface of the cells. These FACS and CELISA assays clearly demonstrate that, in the stable HeLa cell pool, it is possible to restore the cell-surface expression of the HA tag using a peptide inhibitor (e.g. dec-RVKR-cmk) specific for Furin-like PCs.

### Selection of Single Clone Expressing a Hela Furin-Like PCs Specific Chimera

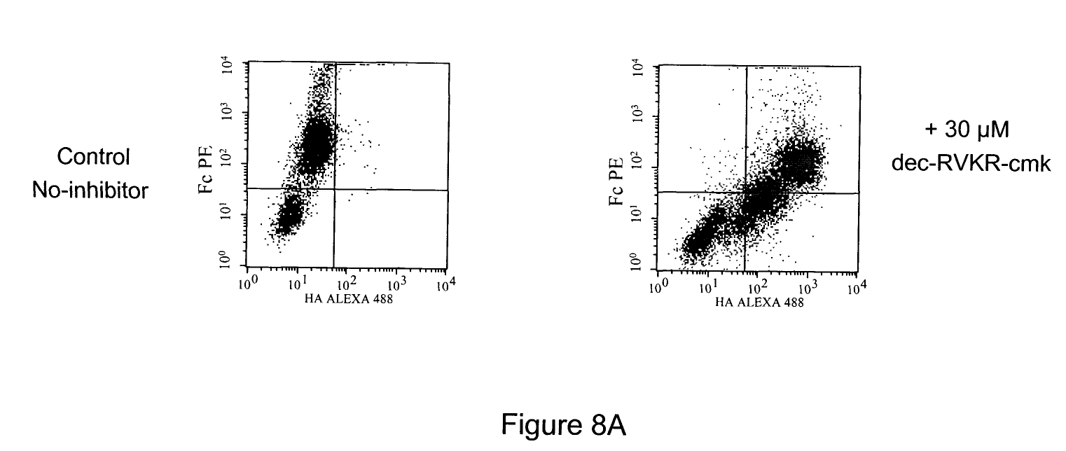

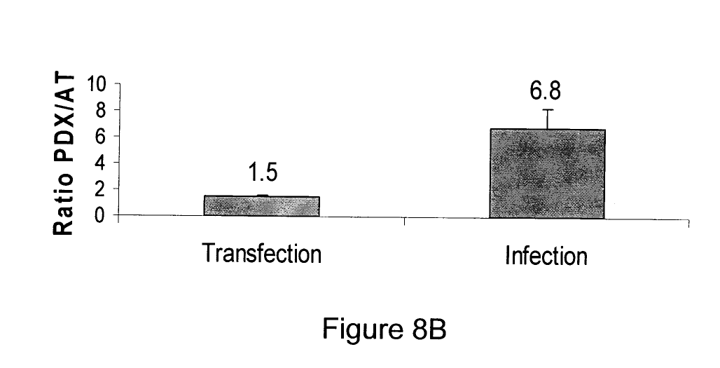

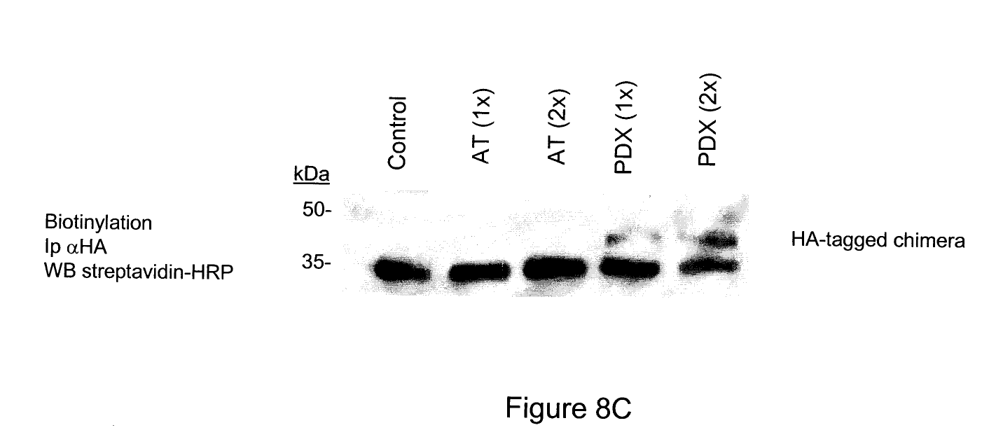

The FACS sorted cells pool was then used to isolate individual clones: 10 clones were isolated of which one (clone PCfur-1.6) expresses a large amount of Fc (i.e. completely cleaved chimera) at the cell surface. This clone was completely converted into an uncleaved form exhibiting the HA-tag at the cell surface in the presence of as little as 30 μM of dec-RVKR-cmk (SEQ ID NO: 35) (FIG. 8A).

### Determination of Specificity of Clone to Constitutively Secreted Furin-Like PCs Inhibitors

Some PCfur-1.6 cells were transfected with an expression vector containing a cDNA coding for either α1-PDX or the α1-AT using lipofectamine as described by the manufacturer (Invitrogen) (less than 20% efficacy), or infected using recombinant adenovirus (Ad) as described (Benjannet et al., 2004) (near 100% efficacy). The expression of α1-PDX (PDX), a known PCs inhibitor, increased the presence of the HA tag at the cell surface. In contrast, expression in parallel cultures of the serpin α1-antitryptin (α1-AT or AT), which does not inhibit PCs, had no effect. As showed in FIG. 8B, a ˜7-fold increase in absorbance was observed from CELISA analyses of cultures expressing recombinant Ad:PDX compared to the ones expressing Ad:AT (infection).

Similar confirmation was obtained using Western blots of cell-surface biotinylated proteins, immunoprecipitated with the HA-antibody and detected on Western using horseraddish peroxidase coupled streptavidin (WB streptavidin-HRP). The Western blot protocol used was a standard one using a monoclonal commercial anti-HA antibody coupled to horseraddish peroxidase (Sigma; used as proposed by the manufacturer) at a final dilution of 1:5000. The blotted proteins were revealed with the ECL plus reagent (Amersham Biosciences), as described by the manufacturer. When the PCfur-1.6 cells were infected with a recombinant adenovirus expressing α1-PDX at 100 (1×) and 200 (2×) million plaque forming units/ml, the presence of the HA tag at the cell surface of these cells could be observed by western blotting of surface biotinylated in FIG. 8C where the stain between the non specific bands at 50 kDa and 35 kDa proteins (FIG. 8C). In contrast, no chimeric HA-tagged chimera could be detected from cultures expressing the serpin α1-antitryptin (α1-AT). This confirms that the HA reappears specifically in the presence of a PCs inhibitor. The utility of the assay was demonstrated with both a peptide (dec-RVKR-cmk (SEQ ID NO: 35)) and a protein (a1-PDX) PC-inhibitors. Small molecules were also identified with the assay (see Example 7).

### Optimization of CELISA Assay

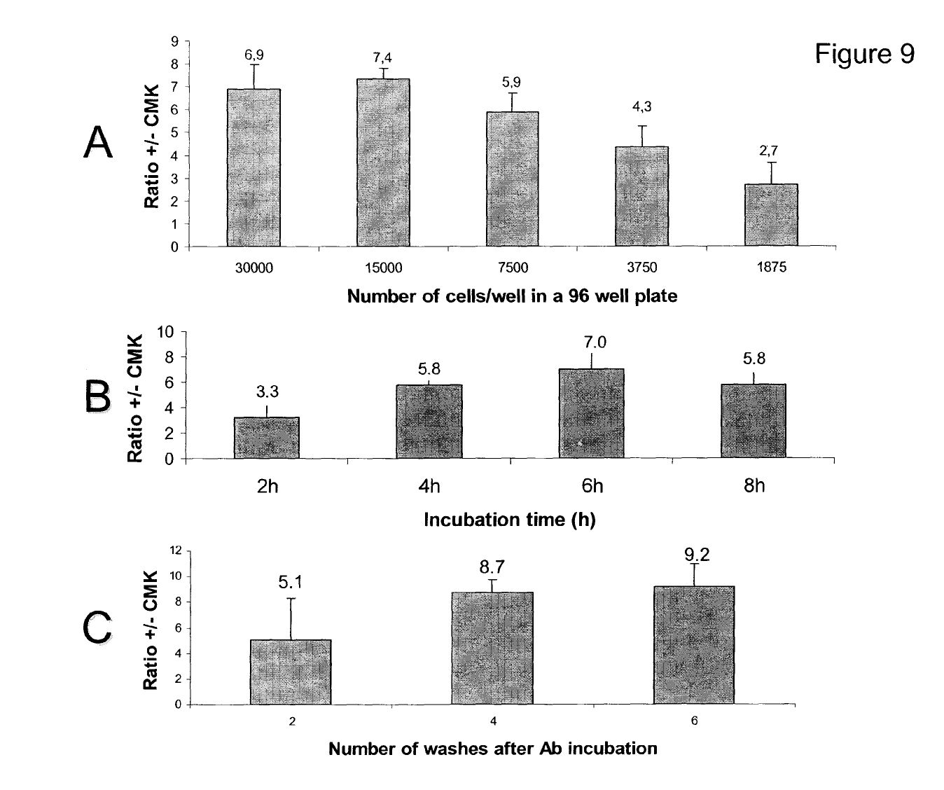

The selected clone PCfur-1.6, was then used to optimize the CELISA assay. The assay was optimized for the number of cells per well (FIG. 9A), the antibody incubation time period (FIG. 9B) and the number of washes (FIG. 9C). The optimization was determined to identify the conditions that yielded the highest ratio of the HA recognition in the presence (+) of 30 μM of the inhibitor dec-RVKR-cmk (CMK) compared to in the absence (−) thereof. The CELISA assay was found to be optimal when using between 15,000-5,000 cells/well; with a 4-8 h HA-antibody incubation time period followed by a minimum of 4 washes.

### Example 2

### Cell-Based Proteinase Inhibitor Screening SKI-1 Inhibitors

A CELISA specific to the SKI-1 was designed using the approach described in Example 1 above, with adaptations. One chimera expressing a bait specific for SKI-1 (IYISRRLL-GTFS (SEQ ID NO: 30)) and a short CT and one chimera with the same bait and the full length-ACE2 CT were constructed as described in Example 1 and used to transfect HuH7 cells.

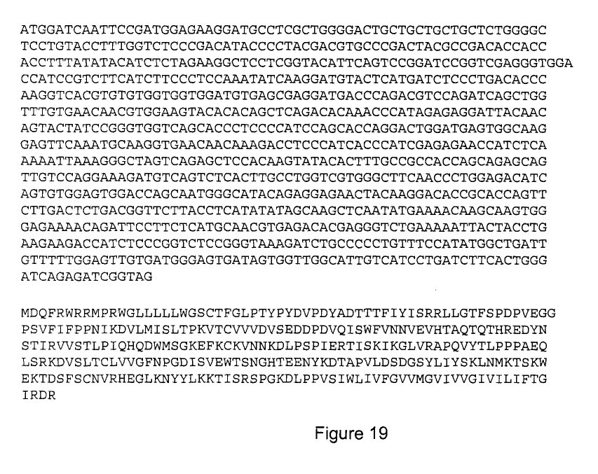

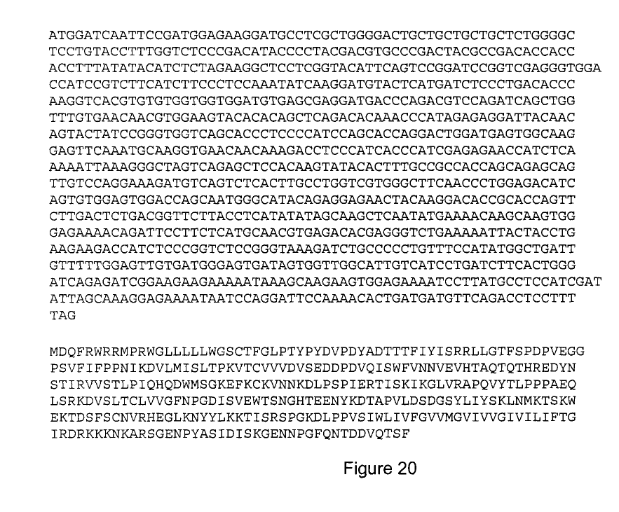

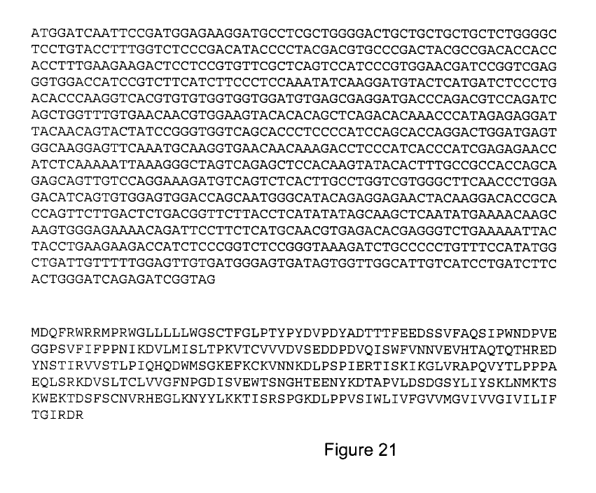

The constructions were obtained by standard PCR and cloning techniques and was made in the model vector pcDNA3 (Invitrogen). The cDNA and amino acid sequences appear in FIGS. 19 and 20.

### Selection of Pools of Cells

HuH7 cells were submitted to two rounds of fluorescence activated cell sorting (FACS) using Alexa488™ as fluorophore with a MoFlo™ cell sorter (Cytomation, Fort Collins, Colo., USA) to obtain pools of cells expressing the Fc (Fc positive) but negative for the HA tag (HA negative) (Data not shown). The presence of Fc tags was the sign that cleavage by SKI-1 PCs at the bait sequence IYISRRLL← GTFS (SEQ ID NO: 30) had released the HA tag.

### Detection of Effect of Convertase Inhibitor on Tag Appearance on Cell Surface

This isolated pool of cells expressing chimera with a bait specific for SKI-1 (IYISRRLL-GTFS (SEQ ID NO: 30)) was incubated for 6 h with 60 μM of dec-YISRRLL-cmk (SEQ ID NO: 42) and submitted to FACS analysis. A reversion of the level of the chimeric protein to that of the uncleaved form (i.e., re-appearance of the HA tag at the cell surface) was observed (Data not shown).

The specificity of the cleavage site was demonstrated in CHO cells devoid of or expressing SKI-1. Using the chimera containing a short ACE2-CT sequence, a 3-fold higher HA tag signal was observed at the cell surface in absence of SKI-1 (Data not shown). The cleavage site specificity was also tested using a chimera harbouring the full length ACE2-CT in human liver HuH7 cells overexpressing a SKI-1 inhibitor, the prosegment R134E mutant (Pullikotil et al, 2004).

### Example 3

### Cell-Based Aspartic Protease BACE Inhibitors Screening

A CELISA specific to BACE was designed using the approach described in Example 1 above, with adaptations. Two chimeras mimicking the Swedish mutation in β-amyloid precursor protein βAPP (FIG. 5) were constructed as described in Example 1 using two sequences known to be cleavable by BACE (KISEVNL-DAE (SEQ ID NO: 33)) and (KISEVNF-EVE (SEQ ID NO: 34)) and the short ACE2-CT segment. Corresponding chimeras with the full length ACE2-CT segment are also constructed since the pH optimum of BACE is acidic and it cleaves bAPP in the TGN or endosomes.

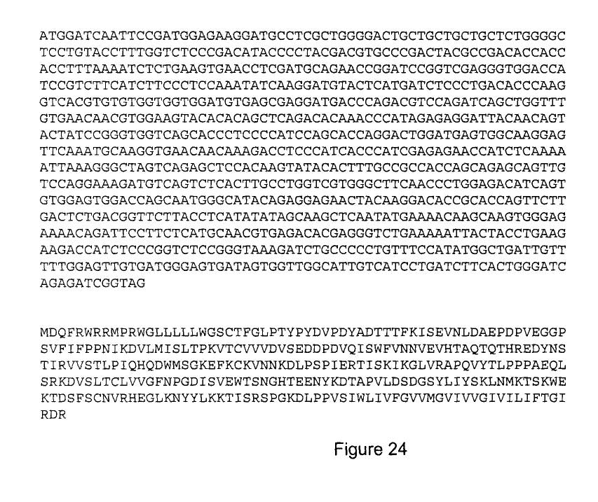

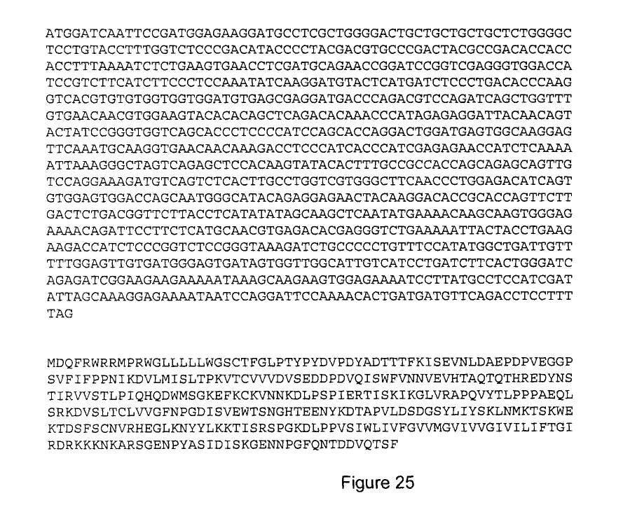

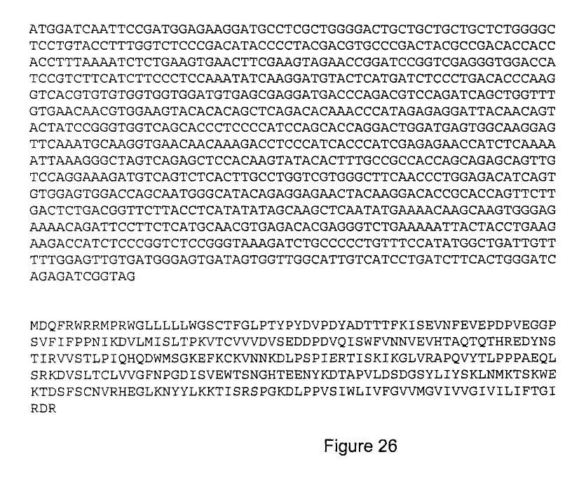

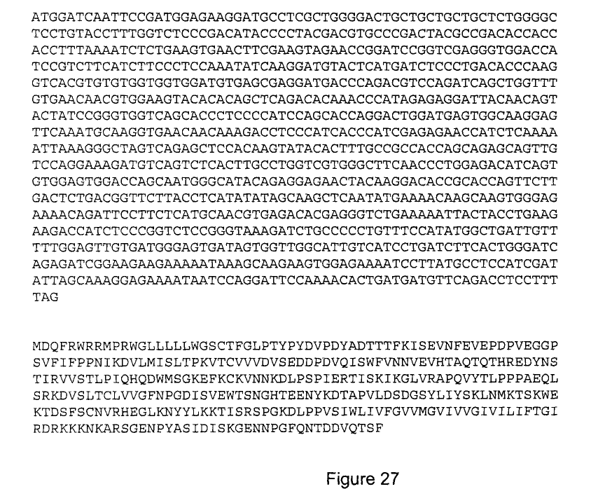

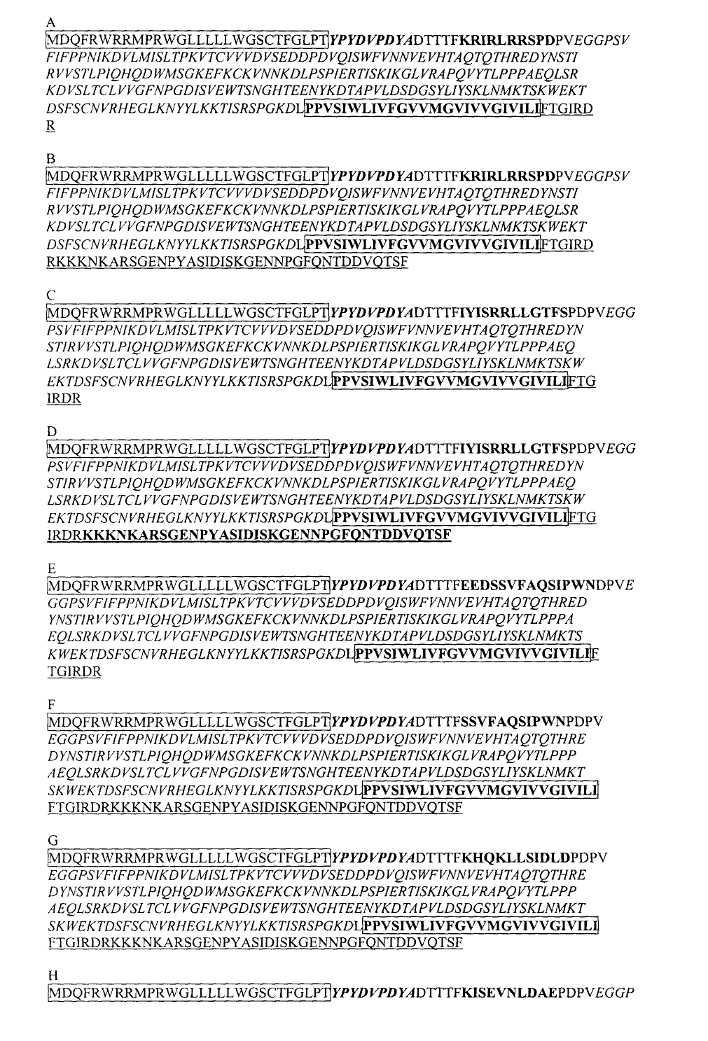

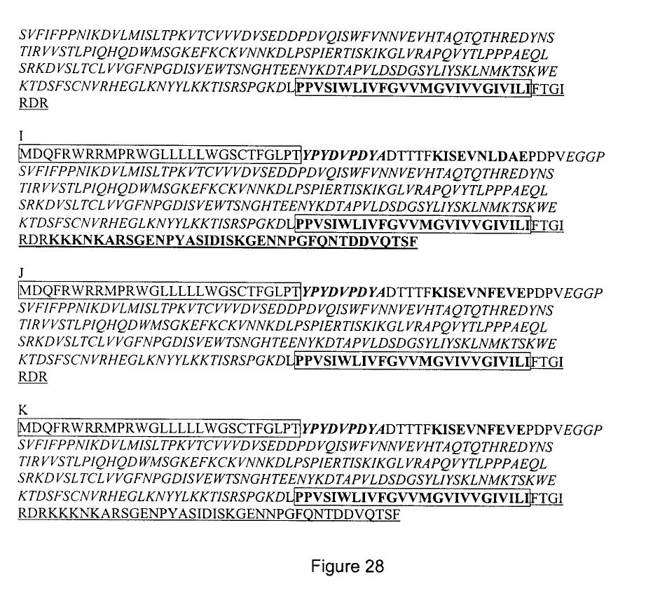

The short CT chimera constructions were obtained by standard PCR and cloning techniques and were made in the model vector pcDNA3 (Invitrogen). The cDNA and amino acid sequences of the short CT chimera appear in FIGS. 24 (BACE) and 26 (BACE mutant). The cDNA and amino acid sequences of the full length CT chimera appear in FIGS. 25 (BACE) and 27 (BACE mutant).

These chimeras are used to transfect mouse Neuro2A. The chimeras are also used to transfect human HuH7 and HeLa cells along with SH-SY5Y neuronal cells.

### Selection of Pools of Cells

Neuro2A cells are submitted to two rounds of fluorescence activated cell sorting (FACS) using Alexa488™ as fluorophore with a MoFlo™ cell sorter (Cytomation, Fort Collins, Colo., USA) to obtain pools of cells expressing the Fc (Fc positive) but negative for the HA tag (HA negative) (Data not shown). The presence of Fc tags is the sign that cleavage by BACE PCs at the bait sequence in the KISEVNL←DAE (SEQ ID NO: 33) or KISEVNF←EVE (SEQ ID NO: 34), depending on the chimera, had released the HA tag.

### Detection of Effect of PC Inhibitor on Tag Appearance on Cell Surface

This isolated pool of cells are incubated for 6 h with 60 μM of JMV2764, a potent PC-inhibitor, and submitted to FACS analysis (Lefranc-Jullien 2005). Reversion of the level of the chimeric protein to that of the uncleaved form (i.e. re-appearance of the HA tag at the cell surface) is observed.

### Example 4

### CELISA NARC-1/PCSK9 Inhibitors Screening

A specific CELISA to the NARC-1/PCSK9 was designed using the approach described in Example 1 above. Chimeras were constructed using sequences known to be cleavable by NARC-1/PCSK9 (SSVFAQ-SIPWN (SEQ ID NO: 31)), or EEDSSVFAQ-SIPWN (SEQ ID NO: 55) combined to full length ACE2-CT segment, and then used to transfect HuH7. Chimeras are also similarly constructed using KHQKLL-SIDLD (SEQ ID NO: 32)). HuH7 was selected amongst 20 cell lines as being one in which the NARC-1/PCSK9 expression level the highest.

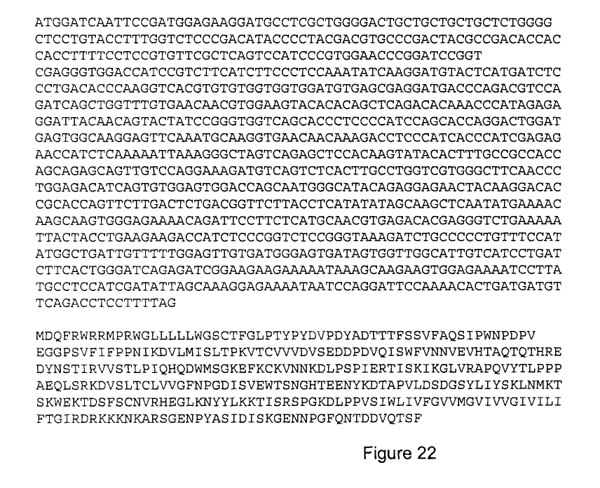

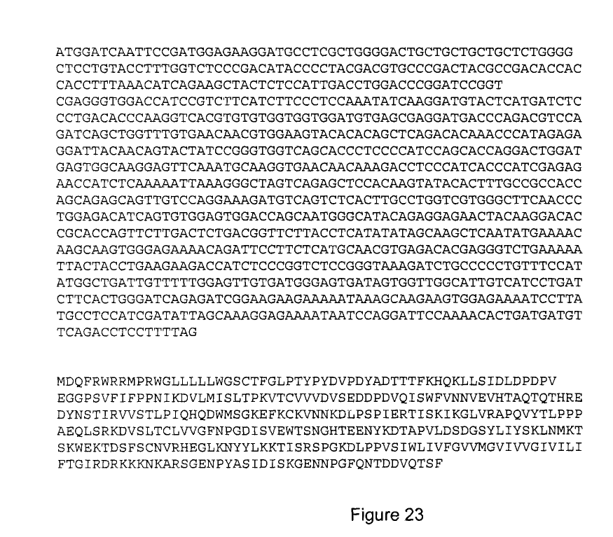

The constructions were obtained by standard PCR and cloning techniques and was made in the model vector pcDNA3 (Invitrogen). The cDNA and amino acid sequences appear in FIGS. 22 and 23.

### Selection of Pools of Cells

HuH7 cells were submitted to two rounds of fluorescence activated cell sorting (FACS) using Alexa488™ as fluorophore with a MoFlo™ cell sorter (Cytomation, Fort Collins, Colo., USA) to obtain pools of cells expressing the lowest amount of the HA tag (Data not shown). This pool of cells is used for the isolation of clones that no longer express or express only a low level of the HA tag at the cell surface (HA low). The presence of Fc tags is the sign that cleavage by NARC-1/PCSK9 PCs at the bait sequence in the SSVFAQ-SIPWN (SEQ ID NO: 31), EEDSSVFAQ-SIPWN (SEQ ID NO: 55) or KHQKLL-SIDLD (SEQ ID NO: 32) released the HA tag.

### Detection of Effect of Convertase Inhibitor on Tag Appearance on Cell Surface

This isolated pool of cells is incubated for 6 h with 5 mM NH₄Cl (see Benjannet J B C 2004), a potent inhibitor of NARC-1/PCSK9 activity and submitted to FACS analysis.

### Detection of Effect of Convertase Activator on Tag Appearance on Cell Surface

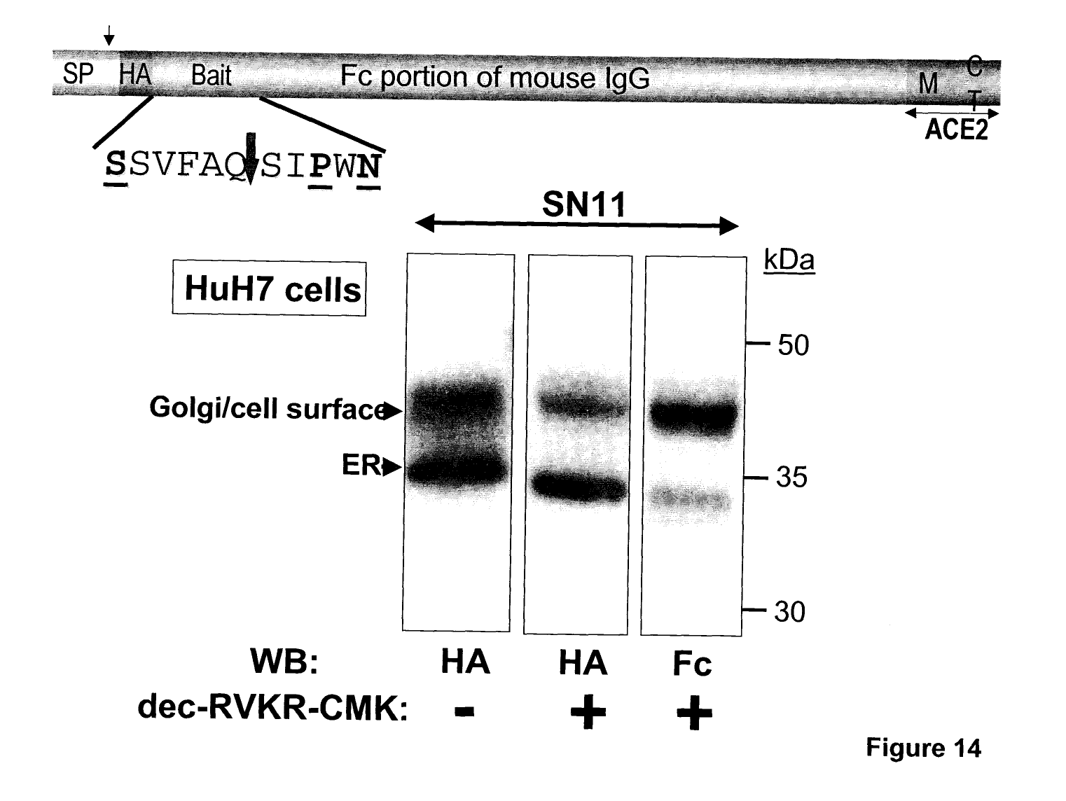

Cells expressing chimera containing the NARC/PCSK9 bait SSVFAQ-SIPWN (SEQ ID NO: 31) (SN11) were analyzed by Western blot. Cells were treated or not for 6 h with 50 μM of dec-RVKR-cmk (SEQ ID NO: 35) to inhibit endogenous Furin-like enzymes and thus indirectly activate NARC/PCSK9. Cells express an approximately equal amounts of ER- (endo H sensitive, not shown) and Golgi-associated (endo H resistant, not shown) SN11 chimera as observed using an HA mAb. In the presence of dec-RVKR-CMK (SEQ ID NO: 35), a substantial decrease in the level of the Golgi-associated HA tag versus the ER-one was observed (FIG. 14). As a control, the level of the Fc immunoreactivity in the presence of dec-RVKR-CMK (SEQ ID NO: 35) was also measured and showed that the Golgi form that lost most of its HA tag, is still very positive for Fc. The Western blot protocol used was a standard one using a monoclonal commercial anti-HA antibody coupled to horseradish peroxidase (Sigma; used as proposed by the manufacturer) at a final dilution of 1:5000. The blotted proteins were revealed with the ECL plus reagent (Amersham Biosciences), as described by the manufacturer.

### Example 5

### CELISA NARC-1/PCSK9-LDLR Coupled Inhibitors Screening

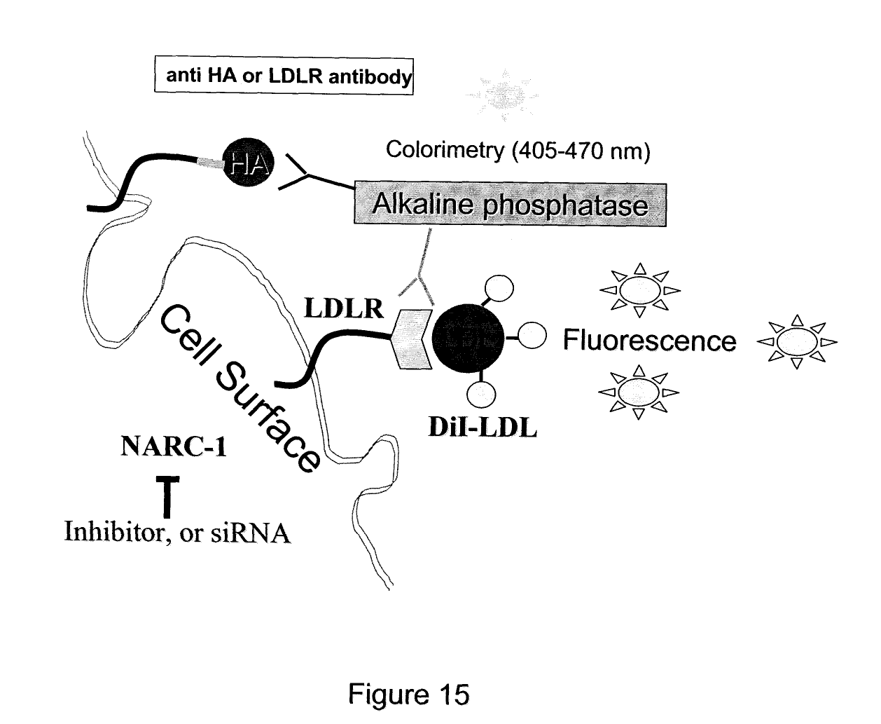

A specific CELISA for the NARC-1/PCSK9 is designed that also affects the accumulation of LDLR at the cell surface (FIG. 15). The chimera expressing a bait specific for NARC-1/PCSK9 (SSVFAQ-SIPWN (SEQ ID NO: 31) is transfected into cells showing both an overexpression of NARC-1/PCSK9 and a low level of LDLR at the cell surface. FACS-selected stable pools of HepG2 or HuH7 cells overexpressing the wild type or the most active natural mutant of NARC-1, namely S127R (EP2003000291025 filed on 2003 Apr. 25 and copending application filed Apr. 23, 2004) and that does not present LDLR at its surface, were selected (Benjannet et al., 2004b). The absence or very low amount of LDLR was tested with fluorogenic LDLR ligand (Dil-LDL) and showed a negligible cell surface binding of the ligand. Of course, other detection methods such as with the use of a monoclonal antibody to LDLR could be used as an alternative. A HepG2 clone expressing a high level of NARC-1/PCSK9 and a low level of LDLR at the cell surface was also isolated. Since siRNA treatment of the cells to reduce NARC-1 expression results in an increase level of LDLR at the cell surface (Benjannet et al., 2004b), inhibitors of NARC-1 will similarly restore the LDLR at the cell surface. Inhibitors of NARC-1 will also in parallel affect the appearance of HA tag from the chimera at the cell surface. The detection of both HA and LDLR at the cell surface could be performed using a variety of assays including CELISA assays and the use of a fluorogenic LDLR ligand or mAB to LDLR coupled to a chemiluminescent probe. Screening could be performed to identify compounds associated to high levels of both the HA tag and the LDLR at the cell surface.

### Example 6

### High Throughput Screening for Furin-Like Inhibitors Using CELISA

The CELISA implemented for Furin was adapted into an automated high throughput screening (HTS) assay to identify Furin inhibitory compounds.

HeLa cells stably and clonally expressing the construct (PCfur-1.6) were used. Liquid nitrogen stocks of these cells were stored so that every vial produced a 15 cm culture dish the day after thawing. They were then passed at a ratio of 1:5 and incubated at 37° C. and 5% CO₂ for three days until confluence. Complete culture medium was composed of Dulbecco's Modified Eagle Medium (D-MEM), high glucose, with L-Glutamine and sodium pyruvate (Invitrogen #11995-065) with 10% FBS. On the first day of the assay, positive (inhibitor: 30 μM dec-RVKR-cmk (SEQ ID NO: 35)) and negative controls, as well as the compounds to be tested, were prepared into complete culture medium, transferred into 384-well clear flat bottom polystyrene tissue culture microplates (Corning, product #3701) at 25 μl per well and kept in the CO₂ incubator for pH equilibration.

Cells were then trypsinized, counted, and resuspended in complete culture medium in order to obtain 320,000 cells per ml, so that 8,000 cells in a 25 μl volume can be added into every well that already contain the inhibitors up to a final volume of 50 μl. They were incubated for 12 h at 37° C. and 5% CO₂.

On the second day of the assay, screening was performed. The following procedures were done at room temperature. First the cells were washed once in 1×PBS and fixed with 20 μl of 4% formalin for 20 minutes. Following this step, they were washed four times with 1×PBS before adding 15 μl of the monoclonal anti-HA peroxidase conjugate, clone HA-7 antibody (sigma, #H 6533) at a 1:6000 dilution for 2 hours. They were washed again, twice, with 1×PBS. Buffer was aspirated before adding 40 μl of the 2,2′-Azino-Bis (3-ethylbenzthiazoline-6-sulfonic acid) substrate (Sigma, #A 3219-100 ml) and incubated for 45 minutes. The substrate formed a blue green reaction product, indicative of the presence of HA at the cell surface, which was read at 405 nm by a plate reader spectrometer.

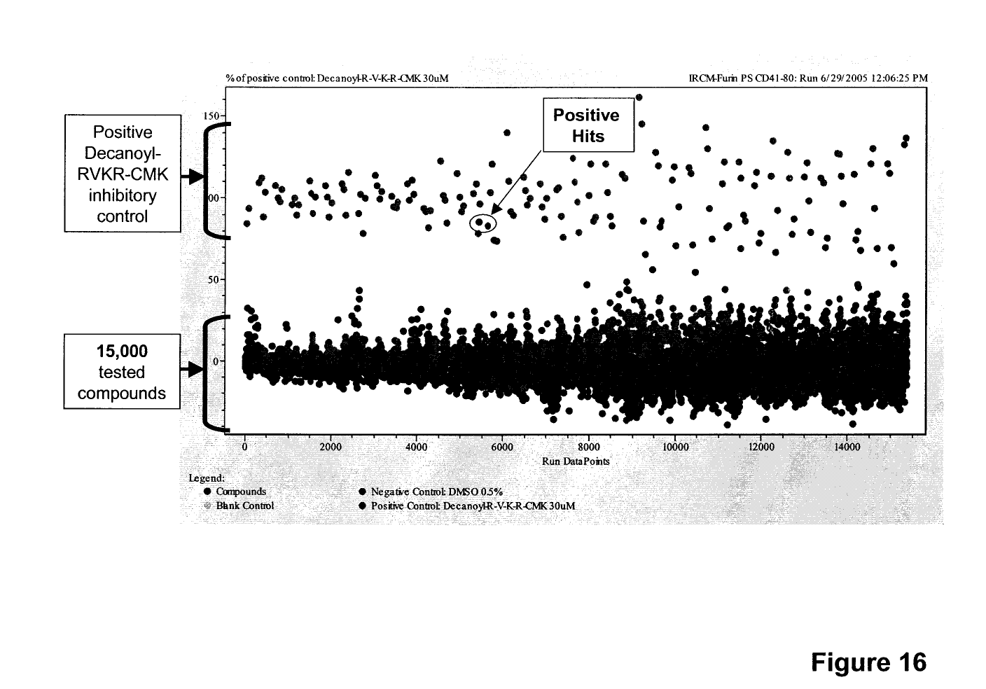

Under these conditions, a Z′ value of 0.6 was obtained with a signal to background ratio of 7. Reproducibility was high and well-well variability was below 10%. Furthermore, this CELISA assay tolerated 0.5% DMSO. The screening identified two hits as may be seen in FIG. 16 with two positive hits.

### Example 7

### Specificity of Constitutively Secreted Furin-Like Inhibitors Identified in CELISA Screening

Compounds including small molecules identified in the screens are further tested for their toxicity to cell viability of primary fibroblasts and endothelial cells. The specificity of these compound is tested in vitro on purified recombinant Furin processing of fluorogenic Boc-RVKR-MCA (SEQ ID NO: 77), and other quench fluorogenic substrates and their kinetics determined. The specificity is also tested ex vivo in CHO-FD11 cells devoid of Furin, but expressing PACE4, PC7 and SKI-1 by measuring the processing for example of PDGF-A or VEGF-C as general substrates for the constitutive PC-like enzymes. Specific bait sequences cleaved by either Furin, PC5, PC7 or PACE4, as shown from in vitro analyses, could also be used (not shown).

### Example 8

### Optimization of Leads

Once inhibitor “leads” are identified, they could be further characterized for affinity, mode of inhibition and specificity using in vitro and ex vivo assays and purified PC enzymes. Hit compounds could be verified by LC mass spectrometry and 10-point titrations could be performed in triplicate on each compound to determine IC50 values (concentration of 50% inhibition). In addition to the screening process itself, expression and purification of modulated candidate PCs for in vitro assays, assay adaptation, and Quantitative Structure-Activity Relationship (QSAR) studies on hits could be performed. Particularly, inhibitors with K is in the nanomolar range are sought.

In principle therefore, the present CELISAs can be applied to any cellular proteinase of the constitutive secretory pathway, the activity of which results in the cleavage of either the chosen chimeras. The isolation of the best inhibitors will find applications in the pharmaceutical industry in diseases such as cancer, Alzheimer's, stress related disorders, dyslipidemias including hypercholesterolemia, atherosclerosis and many others. All these diseases involve the action of one or other of the targeted PCs and/or BACE, the inhibitors of which can be identified by the proposed extended CELISA.

### Example 9

### Identification of SKI-1-Specific Small Molecule Peptidyl Inhibitors—Lassa Viral Glycoprotein-Derived Decanoyl Membrane Permeable Inhibitors

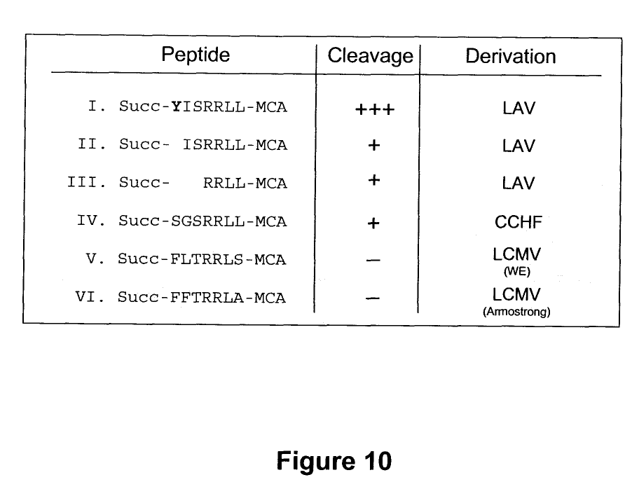

PC activities are routinely assayed using the fluorogenic substrates peptidyl methyl coumarinamides (MCA). The kinetic properties of a number of MCA-peptides based on the Lassa, Crimean Congo hemorrhagic fever (CCHFV) and Lymphocytic Choriomeningitis (LCMV) viral glycoprotein (GPC) recognition SKI-1 site motifs were designed and analyzed as potential in vitro substrates for SKI-1 (FIG. 3). Small molecule specific inhibitors of cellular SKI-1 ex vivo activity were then developed based on the best in vitro Lassa glycoprotein GPC cleavage site (FIG. 10), coupled to an N-terminal decanoyl (dec) membrane permeable moiety and a C-terminal chloromethylketone (cmk) irreversible inhibitor functionality.

### Example 10

### Ex Vivo Inhibition of the SKI-1/S1P Processing of proSREBP-2 and proATF6 with Small Molecule Peptidyl Inhibitors

The ex vivo inhibitory potential of the untested, yet commercially available, 4mer dec-RRLL-cmk (SEQ ID NO: 44) (Bachem, Product N-1885) was then compared to the two synthetic peptides of the present invention, namely the 6mer dec-ISRRLL-cmk (SEQ ID NO: 43) and 7mer dec-YISRRLL-cmk (SEQ ID NO: 42).

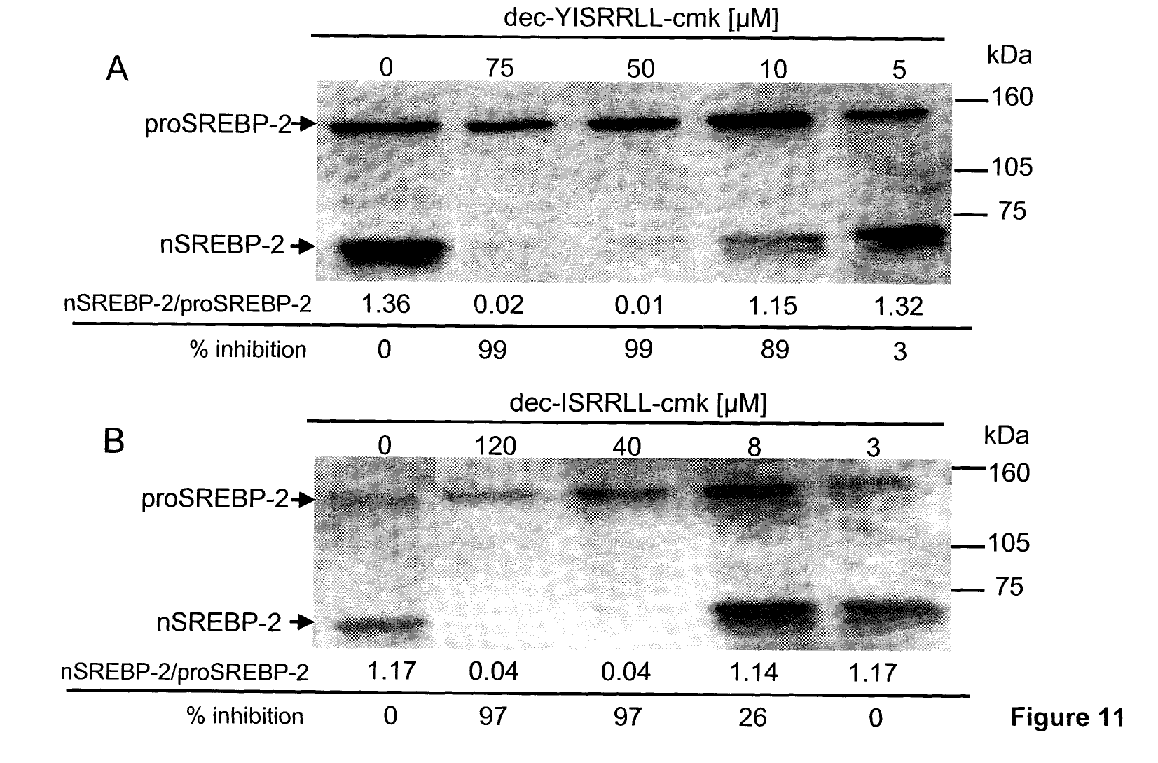

For SREBP-2 analyses, CHOK1 cells were incubated overnight with various concentrations of decanoylated chloromethylketone inhibitors, namely the 7mer-cmk (dec-YISRRLL-cmk (SEQ ID NO: 42), 0-110 μM), 6mer-cmk (dec-ISRRLL-cmk (SEQ ID NO: 43), 0-110 μM), and the 4mer-cmk (dec-RRLL-cmk (SEQ ID NO: 44); Bachem, 0-150 μM). The cell lysates were then analyzed for endogenous SREBP-2. The inhibition of the processing of proSREBP-2 by the 6mer was compared to that of the 7mer cmk-peptides. The data showed that both peptides are potent ex vivo inhibitors of this SKI-1-generated cleavage with an estimated 50% inhibition at ˜7 μM and ˜20 μM for the 7mer-cmk and 6mer-cmk, respectively (FIG. 11). The 7mer was better than the 6mer in this assay.

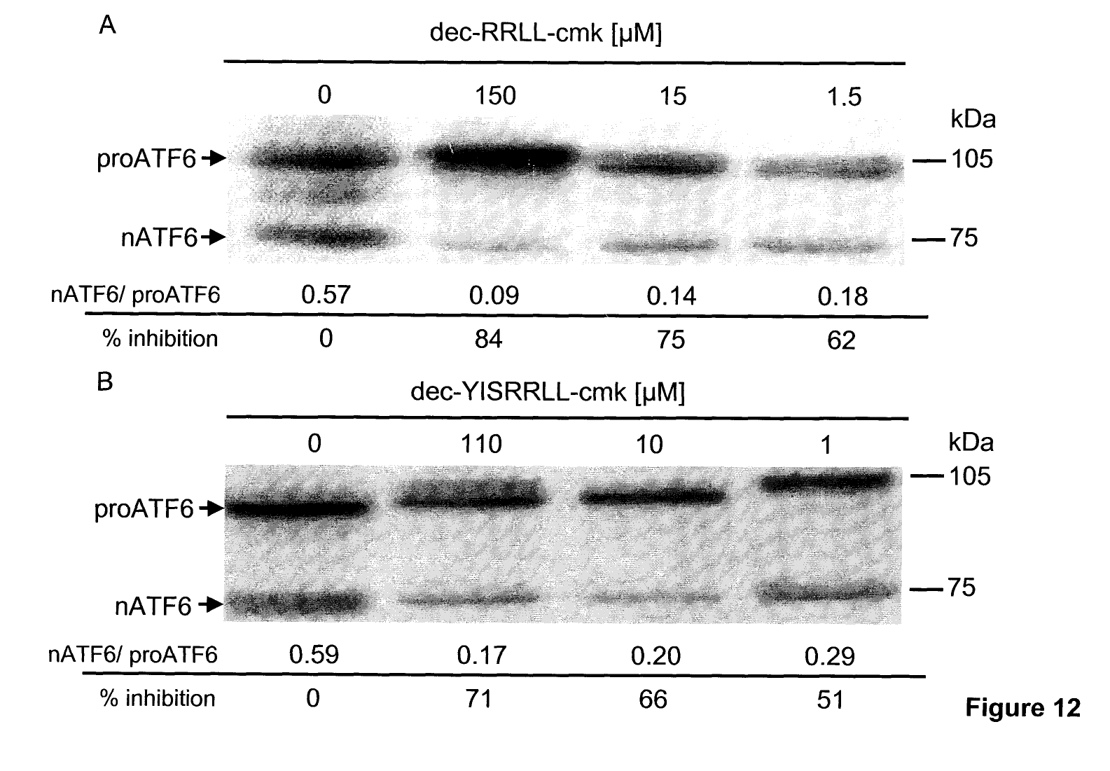

Another experiment showed that both 4mer-cmk and 7mer-cmk peptides are almost equipotent in inhibiting the processing of the overexpressed proATF6 into nATF6 following ER-stress induced by overnight tunicamycin treatment of CHO-cells. It was estimated that 50% inhibition occurs at ≦1 μM of either cmk-peptide (FIG. 12). The dec-4mer-cmk, dec-6mer-cmk and dec-7mer-cmk cell permeable SKI-1 inhibitors are almost equipotent ex vivo.

### Example 11

### Ex Vivo Inhibition of the Furin-Like Processing of proPDGF-A by SKI-1 Small Molecule Peptidyl Inhibitors

The selectivity of the above cmk-peptides for inhibition of SKI-1 was determined by comparing these peptides' ability to inhibit SKI-1-generated processing reactions to their ability to inhibit other PCs-generated processing reactions. The ability of the 7mer, the 4mercmk-peptides to inhibit the processing of the precursor of the platelet derived growth factor propDGF-A into PDGF-A by Furin-like dibasic-specific amino acid PCs was compared to that of the frequently used commercially available Furin-like convertase inhibitor dec-RVKR-cmk (SEQ ID NO: 35).

On day 0, stable PDGF-A-V5 construct in HEK293 cells, were plated on 35 mm plates in (Dulbecco's modified Eagle's medium containing 100 units/ml gentamycin) supplemented with 5% heat inactivated fetal calf serum. On day 1, cells were washed twice with 1× phosphate buffered saline (PBS) and serum free medium was added along with varying concentrations of dec-YISRRLL-cmk (0-110 μM) (SEQ ID NO: 42) or dec-RRLL-cmk (0-150 μM) (SEQ ID NO: 44), incubated overnight. Day 3, medium was analyzed by running the samples on 12% SDS-PAGE and immunoblotted using V5-HRP antibody 1:5000.

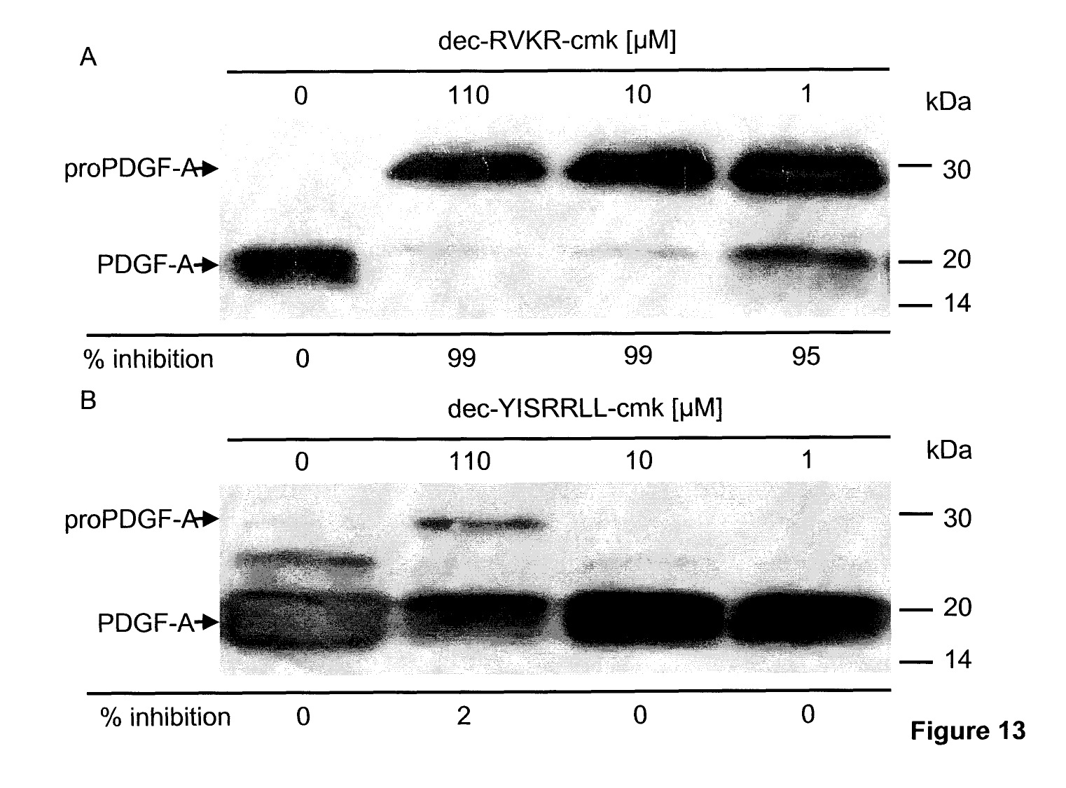

The data showed that in HEK293 cells stably expressing propDGF-A the processing of this precursor by endogenous Furin-like PCs is completely inhibited by ˜3 μM dec-RVKR-cmk (SEQ ID NO: 35), whereas it would take >100 μM to inhibit less than 2% of this reaction by the 7mer dec-YISRRLL-cmk (SEQ ID NO: 42) or the 4mer dec-RRLL-cmk (SEQ ID NO: 44) (FIG. 13).

Although the present invention has been described hereinabove by way of specific embodiments thereof, it can be modified, without departing from the spirit and nature of the subject invention as defined in the appended claims.

### REFERENCES

1. Abifadel, M., Varret, M., Rabes, J. P., Allard, D., Ouguerram, K., Devillers, M., Cruaud, C., Benjannet, S., Wickham, L., Erlich, D., Derre, A., Villeger, L., Farnier, M., Beucler, I., Bruckert, E., Chambaz, J., Chanu, B., Lecerf, J. M., Luc, G., Moulin, P., Weissenbach, J., Prat, A., Krempf, M., Junien, C., Seidah, N. G., and Boileau, C. (2003). Mutations in PCSK9 cause autosomal dominant hypercholesterolemia. Nat. Genet. 34, 154-156.
2. Anderson, E. D., Thomas, L., Hayflick, J. S., and Thomas, G. (1993). Inhibition of HIV-1 gp160-dependent membrane fusion by a Furin-directed alpha 1-antitrypsin variant. J. Biol. Chem. 268, 24887-24891.
3. Belkhiri A, Lytvyn V, Guilbault C, Bourget L, Massie B, Nagler D K, Menard R. (2002) A noninvasive cell-based assay for monitoring proteolytic activity within a specific subcellular compartment. Anal Biochem. July 15; 306(2):237-46.
4. Benjannet, S., Cromlish, J. A., Diallo, K., Chretien, M., and Seidah, N. G. (2004a). The metabolism of beta-amyloid converting enzyme and beta-amyloid precursor protein processing. Biochem. Biophys. Res. Commun. 325, 235-242.
5. Benjannet, S., Elagoz, A., Wickham, L., Mamarbachi, M., Munzer, J. S., Basak, A., Lazure, C., Cromlish, J. A., Sisodia, S., Checker, F., Chretien, M., and Seidah, N. G. (2001). Post-translational processing of beta-secretase (beta-amyloid-converting enzyme) and its ectodomain shedding. The pro- and transmembrane/cytosolic domains affect its cellular activity and amyloid-beta production. J. Biol. Chem. 276, 10879-10887.
6. Benjannet, S., Rhainds, D., Essalmani, R., Mayne, J., Wickham, L., Jin, W., Asselin, M. C., Hamelin, J., Varret, M., Allard, D., Trillard, M., Abifadel, M., Tebon, A., Attie, A. D., Rader, D. J., Boileau, C., Brissette, L., Chretien, M., Prat, A., and Seidah, N. G. (2004b). NARC-1/PCSK9 and Its Natural Mutants: zymogen cleavage and effects on the low density lipoprotein (LDL) receptor and LDL cholesterol. J Biol. Chem. 279, 48865-48875.
7. Benjarinet, S., Savaria, D., Laslop, A., Munzer, J. S., Chretien, M., Marcinkiewicz, M., and Seidah, N. G. (1997). Alpha1-antitrypsin Portland inhibits processing of precursors mediated by proprotein convertases primarily within the constitutive secretory pathway. J. Biol. Chem. 272, 26210-26218.
8. Bergeron, E., Vincent, M. J., Wickham, L., Hamelin, J., Basak, A., Nichol, S. T., Chretien, M., and Seidah, N. G. (2005). Implication of proprotein convertases in the processing and spread of severe acute respiratory syndrome coronavirus. Biochem. Biophys. Res. Commun. 326, 554-563.
9. Brown, M. S. and Goldstein, J. L. (1999). A proteolytic pathway that controls the cholesterol content of membranes, cells, and blood. Proc. Natl. Acad. Sci. U.S.A. 96, 11041-11048.
10. Cohen, J., Pertsemlidis, A., Kotowski, I. K., Graham, R., Garcia, C. K., and Hobbs, H. H. (2005). Low LDL cholesterol in individuals of African descent resulting from frequent nonsense mutations in PCSK9. Nat. Genet. 37, 161-165.
11. Jadot, M. et al., (1992) J. Biol. Chem. 267, 11069.
12. Jutras, I., Seidah, N. G., and Reudelhuber, T. L. A predicted alpha-helix mediates targeting of the proprotein convertase PC1 to the regulated secretory pathway. J. Biol. Chem., 275: 40337-40343, 2000.
13. Khatib, A. M., Siegfried, G., Chretien, M., Metrakos, P., and Seidah, N. G. (2002). Proprotein convertases in tumor progression and malignancy: novel targets in cancer therapy. Am. J Pathol. 160, 1921-1935.
14. Khatib, A. M., Siegfried, G., Prat, A., Luis, J., Chretien, M., Metrakos, P., and Seidah, N. G. (2001). Inhibition of proprotein convertases is associated with loss of growth and tumorigenicity of HT-29 human colon carcinoma cells: importance of insulin-like growth factor-1 (IGF-1) receptor processing in IGF-1-mediated functions. J. Biol. Chem. 276, 30686-30693.
15. Lefranc-Jullien S, Lisowski V, Hernandez J F, Martinez J, Checker F. (2005). Design and characterization of a new cell-permeant inhibitor of the beta-secretase BACE1. Br J Pharmacol. 145, 228-35.
16. Lopez-Perez E., Seidah N. G., and Checker F. (1999). Proprotein convertase activity contributes to the processing of the Alzheimer's beta-amyloid precursor protein in human cells: evidence for a role of the prohormone convertase PC7 in the constitutive alpha-secretase pathway. J. Neurochem. 73, 2056-2062.
17. Lopez-Perez E., Zhang Y., Frank S. J., Creemers J., Seidah N., and Checker F. (2001). Constitutive alpha-secretase cleavage of the beta-amyloid precursor protein in the Furin-deficient LoVo cell line: involvement of the pro-hormone convertase 7 and the disintegrin metalloprotease ADAM10. J. Neurochem. 76, 1532-1539.
18. Oh M, Kim S Y, Oh Y S, Choi D Y, Sin H J, Jung I M, Park W J. (2004) Cell-based assay for beta-secretase activity. Bioorg Med Chem Lett. 14(24): 6071-4.
19. Pasquato A, Pullikotil P, Asselin M A, Vacatello M, Paolillo L, Ghezzo F. Basso F, Di Bello C, Dettin M, and Seidah N G (2006). The proprotein convertase ski-1/s1p in vitro analysis of lassa virus glycoprotein-derived substrates and ex vivo validation of irreversible peptide inhibitors. J. Biol. Chem. 281(33), 23471-81.
20. Pullikotil, P., Vincent, M., Nichol, S. T., and Seidah, N. G. (2004). Development of protein-based inhibitors of the proprotein of convertase SKI-1/S1P: processing of SREBP-2, ATF6, and a viral glycoprotein. J. Biol. Chem. 279, 17338-17347.
21. Seidah, N. G. (2002). Cellar limited proteolysis of precursor proteins and peptides. In Co- and post-translational proteolysis of proteins, R. E. Dalbey and D. S. Sigman, eds. (San Diego: Academic Press), pp. 237-259.
22. Seidah, N. G., Benjannet, S., Wickham, L., Marcinkiewicz, J., Jasmin, S. B., Stifani, S., Basak, A., Prat, A., and Chretien, M. (2003). The secretory proprotein convertase neural apoptosis-regulated convertase 1 (NARC-1): liver regeneration and neuronal differentiation. Proc. Natl. Acad. Sci. U.S. A 100, 928-933.
23. Seidah, N. G. and Chretien, M. (1997). Eukaryotic protein processing: endoproteolysis of precursor proteins. Curr. Opin. Biotechnol. 8, 602-607.
24. Seidah, N. G. and Chretien, M. (1999). Proprotein and prohormone convertases: a family of subtilases generating diverse bioactive polypeptides. Brain Res. 848, 45-62.
25. Seidah, N. G., Mbikay, M., Marcinkiewicz, M., and Chretien, M. (1998). The mammalian precursor convertases: paralogs of the subtilisin/kexin family of calcium-dependent serine proteinases. In Proteolytic and cellular mechanisms in prohormone and neuropeptide precursor processing, V. Y. Hook, ed. (Georgetown, Tex.: R.G. Landes company), pp. 49-76.
26. Seidah, N. G., Mowla, S. J., Hamelin, J., Mamarbachi, A. M., Benjannet, S., Toure, B. B., Basak, A., Munzer, J. S., Marcinkiewicz, J., Zhong, M., Barale, J. C., Lazure, C., Murphy, R. A., Chretien, M., and Marcinkiewicz, M. (1999). Mammalian subtilisin/kexin isozyme SKI-1: A widely expressed proprotein convertase with a unique cleavage specificity and cellular localization. Proc. Natl. Acad. Sci. U.S. A 96, 1321-1326.
27. Seidah, N. G. and Prat, A. (2002). Precursor convertases in the secretory pathway, cytosol and extracellular milieu. Essays Biochem. 38, 79-94.
28. Siegfried, G., Basak, A., Cromlish, J. A., Benjannet, S., Marcinkiewicz, J., Chretien, M., Seidah, N. G., and Khatib, A. M. (2003a). The secretory proprotein convertases Furin, PC5, and PC7 activate VEGF-C to induce tumorigenesis. J. Clin. Invest 111, 1723-1732.
29. Siegfried, G., Basak, A., Prichett-Pejic, W., Scamuffa, N., Ma, L., Benjannet, S., Veinot, J. P., Chretien, M., Seidah, N. G., and Khatib, A. M. Regulation of the stepwise proteolytic cleavage and secretion of PDGF B by the proprotein convertases. Oncogene (in press). 2005.
30. Siegfried, G., Khatib, A. M., Benjannet, S., Chretien, M., and Seidah, N. G. (2003b). The proteolytic processing of pro-platelet-derived growth factor-A at RRKR(86) by members of the proprotein convertase family is functionally correlated to platelet-derived growth factor-A-induced functions and tumorigenicity. Cancer Res. 63, 1458-1463.
31. Toure, B. B., Munzer, J. S., Basak, A., Benjannet, S., Rochemont, J., Lazure, C., Chretien, M., and Seidah, N. G. (2000). Biosynthesis and enzymatic characterization of human SKI-1/S1P and the processing of its inhibitory prosegment. J. Biol. Chem. 275, 2349-2358.
32. Turk, B. E., Huang, L. L., Piro, E. T., Cantley, L. C. (2001). Determination of protease cleavage site motifs using mixture-based oriented peptide libraries. Nat. Biotechnol. 19, 661-667.
33. Wickham, L., Benjannet, S., Marcinkiewicz, E., Chretien, M., and Seidah, N. G. (2005). Beta-amyloid protein converting enzyme 1 and brain-specific type II membrane protein BRI3: binding partners processed by Furin. J. Neurochem. 92, 93-102.
34. Wiley, J. & Sons, Inc. Current Protocols in Molecular Biology (On line) http://www.mrw.interscience.wiley.com/cp/cpmb/cpmb_contents_fs.html.
35. Zhong, M., Munzer, J. S., Basak, A., Benjannet, S., Mowla, S. J., Decroly, E., Chretien, M., and Seidah, N. G. (1999). The prosegments of Furin and PC7 as potent inhibitors of proprotein convertases. In vitro and ex vivo assessment of their efficacy and selectivity. J. Biol. Chem. 274, 33913-33920.

1. A chimeric protein comprising in sequence a signal peptide, a first amino acid tag, a proteinase bait, a second amino acid tag, a transmembrane domain and a cytosolic domain, wherein the cytosolic (CT) domain comprises a sequence able to recycle the protein from the cellular membrane to endosomes.

2. The chimeric protein of claim 1, wherein the second tag is an immunoglobulin Fc fragment of at least 20 amino acid residues.

3. The chimeric protein of claim 1, wherein the CT comprises a Y-X-X-hydrophobic motif, wherein X is any amino acid.

4. The chimeric protein of claim 1, wherein the CT domain is the full-length CT of ACE2 as set forth in SEQ ID NO: 73.

5. The chimeric protein of claim 1, wherein the first tag is a hemaglutinin A domain (HA).

6. The chimeric protein of claim 1, wherein the bait has a length of 9 to 30 amino acid residues.

7. The chimeric protein of claim 1, wherein the bait comprises an amino acid sequence as set forth in the formula (K/R)-(X)n-(K/R), where n=0 (SEQ ID NO: 1), 2 (SEQ ID NO: 2), 4 (SEQ ID NO: 3) or 6 (SEQ ID NO: 4) and X is any amino acid.

8. The chimeric protein of claim 7, wherein the bait comprises an amino acid sequence as set forth in KRIRLRRSPD (SEQ ID NO: 29).

9. The chimeric protein of claim 8, the amino acid sequence of which is as set forth in SEQ ID NO: 48.

10. The chimeric protein of claim 8, encoded by a nucleotide sequence as set forth in SEQ ID NO: 47.

11. The chimeric protein of claim 7, wherein the bait comprises KRIRLRRLPD (SEQ ID NO: 76).

12. The chimeric protein of claim 1, wherein the bait comprises an amino acid sequence as set forth in the formula R-X-(L/V)-Z, wherein X is any amino acid and Z is any amino acid except E, D, C, P and V (SEQ ID NO: 9).

13. The chimeric protein of claim 12, wherein the bait comprises an amino acid sequence as set forth in IYISRRLLGTFS (SEQ ID NO: 30).

14. The chimeric protein of claim 12, the amino acid sequence of which is as set forth in SEQ ID NO: 52.

15. The chimeric protein of claim 12, encoded by a nucleotide sequence as set forth SEQ ID NO: 51.

16. The chimeric protein of claim 1, wherein the bait comprises an amino acid sequence as set forth in VFAQSIP (SEQ ID NO: 10).

17. The chimeric protein of claim 1, wherein the bait comprises an amino acid sequence as set forth in SSVFAQSIPWN (SEQ ID NO: 31).

18. The chimeric protein of claim 1, wherein the bait comprises an amino acid sequence as set forth in KHQKLLSIDLD (SEQ ID NO: 32).

19. The chimeric protein of claim 17, the amino acid sequence of which is as set forth in SEQ ID NO: 57.

20. The chimeric protein of claim 17, encoded by a nucleotide sequence as set forth in SEQ ID NO: 56.

21. The chimeric protein of claim 18, the amino acid sequence of which is as set forth in SEQ ID NO: 59.

22. The chimeric protein of claim 18, encoded by a nucleotide sequence as set forth in SEQ ID NO: 58.

23. The chimeric protein of claim 1, wherein the bait comprises an amino acid sequence as set forth in KISEVNLDAE (SEQ ID NO: 33).

24. The chimeric protein of claim 1, wherein the bait comprises an amino acid sequence as set forth in KISEVNFEVE (SEQ ID NO: 34).

25. The chimeric protein of claim 23, the amino acid sequence of which is as set forth in SEQ ID NO: 63.

26. The chimeric protein of claim 23, encoded by a nucleotide sequence as set forth in SEQ ID NO: 62.

27. The chimeric protein of claim 24, the amino acid sequence of which is as set forth in SEQ ID NO: 67.

28. The chimeric protein of claim 24, encoded by a nucleotide sequence as set forth in SEQ ID NO: 66.

29. A cell line stably expressing the chimeric protein of claim 1 and expressing a proteinase able to cleave the bait of the chimeric protein.

30. A cell line stably expressing the chimeric protein of claim 7, wherein the cell line is a HeLa cell line.

31. A cell line stably expressing the chimeric protein of claim 11, wherein the cell line is a human lung epithelial A549 cell line.

32. A cell line stably expressing the chimeric protein of claim 12, wherein the cell line is a HuH7 cell line.

33. A cell line stably expressing the chimeric protein of claim 16, wherein the cell line is a HuH7 cell line.

34. The cell line of claim 33 wherein the cell line overexpresses NARC1/PCSK9 or the S127R mutated form of the NARC-1/PCSK9; and expresses a low level of LDLR at the cell surface.

35. The cell line stably expressing the chimeric protein of claim 23, wherein the cell line is a Neuro2A cell line.

36. A cell-based assay for identifying a constitutively secreted proteinase modulator, which comprises the steps of:
(a) providing the cell line of claim 29;
(b) measuring the presence of the first amino acid tag at the cell surface in the presence of a candidate modulator and in the absence thereof, whereby a difference in the level of detection of the tag in the presence of the candidate modulator as compared to in the absence thereof is an indication that the candidate is a constitutively secreted proteinase modulator.

37. The assay of claim 36 for identifying a constitutively secreted proteinase inhibitor, wherein the candidate modulator is a candidate inhibitor whereby a higher level of detection of the first amino acid tag in the presence of the candidate inhibitor as compared to in the absence thereof is an indication that the candidate is a constitutively secreted proteinase inhibitor.

38. The assay of claim 36 for identifying a constitutively secreted proteinase activator, wherein the candidate proteinase modulator is a candidate proteinase activator and wherein the cell line expresses a ratio of first amino acid tag:second amino acid tag between about 10:90 and about 30:70, whereby a lower level of detection of the first amino acid tag in the presence of the candidate activator as compared to in the absence thereof is an indication that the candidate is a constitutively secreted proteinase activator.

39. The assay of claim 36, wherein the assay is performed on an intact cell.

40. The assay of claim 36, wherein the assay is performed using a cell lysate.

41. A cell-based assay for identifying a constitutively secreted proteinase modulator, which comprises the steps of:
(a) providing the cell line of claim 29;
(b) measuring the presence of the first amino acid tag in the cell culture supernatant in the presence of a candidate modulator and in the absence thereof, whereby a difference in the level of detection of the tag in the presence of the candidate modulator as compared to in the absence thereof is an indication that the candidate is a constitutively secreted proteinase modulator.

42. The assay of claim 41 for identifying a constitutively secreted proteinase activator, wherein the candidate proteinase modulator is a candidate proteinase activator whereby a higher level of detection of the first amino acid tag in the supernatant in the presence of the candidate activator as compared to in the absence thereof is an indication that the candidate is a constitutively secreted proteinase activator.

43. The assay of claim 36, wherein the presence of the first amino acid tag is directly measurable using fluorometry.

44. The assay of claim 36, wherein the presence of the first amino acid tag is measurable by measurement of the activity of an enzyme on a substrate.

45. The assay of claim 44, wherein the enzyme is alkaline phosphatase.

46. The assay of claim 36, wherein the presence of the first amino acid tag is measurable by the binding of a ligand to the first amino acid tag.

47. A SKI-1 convertase substrate as set forth in Succ-YISRRLL-MCA (SEQ ID NO: 36).

48. A SKI-1 convertase inhibitor as set forth in dec-YISRRLL-cmk (SEQ ID NO: 42).

49. A SKI-1 convertase inhibitor as set forth in dec-ISRRLL-cmk (SEQ ID NO: 43).

50. A SKI-1 convertase substrate as set forth in Succ-ISRRLL-MCA (SEQ ID NO: 37).

51. (canceled)
